SEQUENCE LISTING (2 sequences)

SEQ ID NO: 1 - DNA, 1587 bp, Homo sapiens
  CDS (124)..(1572)
gggcgaggggcggacaccggagagacacgggaaaggggtcgggacaggagcacgtggctcagacaccgacgccgggaggccgcagaccccggacgtgtcaggcatccccgcaggcccggagcgatggcagccttgatgaccccgggaaccggggccccacccgcgcctggtgacttctccggggaagggagccagggacttcccgacccttcgccagagcccaagcagctcccggagctgatccgcatgaagcgagacggaggccgcctgagcgaagcggacatcaggggcttcgtggccgctgtggtgaatgggagcgcgcagggcgcacagatcggggccatgctgatggccatccgacttcggggcatggatctggaggagacctcggtgctgacccaggccctggctcagtcgggacagcagctggagtggccagaggcctggcgccagcagcttgtggacaagcattccacagggggtgtgggtgacaaggtcagcctggtcctcgcacctgccctggcggcatgtggctgcaaggtgccaatgatcagcggacgtggtctggggcacacaggaggcaccttggataagctggagtctattcctggattcaatgtcatccagagcccagagcagatgcaagtgctgctggaccaggcgggctgctgtatcgtgggtcagagtgagcagctggttcctgcggacggaatcctatatgcagccagagatgtgacagccaccgtggacagcctgccactcatcacagcctccattctcagtaagaaactcgtggaggggctgtccgctctggtggtggacgttaagttcggaggggccgccgtcttccccaaccaggagcaggcccgggagctggcaaagacgctggttggcgtgggagccagcctagggcttcgggtcgcggcagcgctgaccgccatggacaagcccctgggtcgctgcgtgggccacgccctggaggtggaggaggcgctgctctgcatggacggcgcaggcccgccagacttaagggacctggtcaccacgctcgggggcgccctgctctggctcagcggacacgcggggactcaggctcagggcgctgcccgggtggccgcggcgctggacgacggctcggcccttggccgcttcgagcggatgctggcggcgcagggcgtggatcccggtctggcccgagccctgtgctcgggaagtcccgcagaacgccggcagctgctgcctcgcgcccgggagcaggaggagctgctggcgcccgcagatggcaccgtggagctggtccgggcgctgccgctggcgctggtgctgcacgagctcggggccgggcgcagccgcgctggggagccgctccgcctgggggtgggcgcagagctgctggtcgacgtgggtcagaggctgcgccgtgggaccccctggctccgcgtgcaccgggacggccccgcgctcagcggcccgcagagccgcgccctgcaggaggcgctcgtactctccgaccgcgcgccattcgccgccccctcgcccttcgcagagctcgttctgccgccgcagcaataaagctcctttgccgcg

SEQ ID NO: 2 - protein, 482 aa, Homo sapiens
MAALMTPGTGAPPAPGDFSGEGSQGLPDPSPEPKQLPELIRMKRDGGRLSEADIRGFVAAVVNGSAQGAQIGAMLMAIRLRGMDLEETSVLTQALAQSGQQLEWPEAWRQQLVDKHSTGGVGDKVSLVLAPALAACGCKVPMISGRGLGHTGGTLDKLESIPGFNVIQSPEQMQVLLDQAGCCIVGQSEQLVPADGILYAARDVTATVDSLPLITASILSKKLVEGLSALVVDVKFGGAAVFPNQEQARELAKTLVGVGASLGLRVAAALTAMDKPLGRCVGHALEVEEALLCMDGAGPPDLRDLVTTLGGALLWLSGHAGTQAQGAARVAAALDDGSALGRFERMLAAQGVDPGLARALCSGSPAERRQLLPRAREQEELLAPADGTVELVRALPLALVLHELGAGRSRAGEPLRLGVGAELLVDVGQRLRRGTPWLRVHRDGPALSGPQSRALQEALVLSDRAPFAAPSPFAELVLPPQQ

(19) United States
(12) Patent Application Publication
(10) Pub. No.: US 2003/0091990 A1
(43) Pub. Date: May 15, 2003

(54) Methods and compositions involving thymidine phosphorylase as a marker for HIV infection, aids progression, and drug resistance

(76) Inventors: Miles W. Cloyd, Galveston, TX (US); Kyeongeun Lee, Galveston, TX (US); David Paar, Galveston, TX (US); Jenny Chen, Philadelphia, PA (US); Liqiang Wang, Galveston, TX (US)

Correspondence Address: Gina N. Shishima, Fulbright & Jaworski L.L.P., Suite 2400, 600 Congress Avenue, Austin, TX, 78701, US

(21) Appl. No.: 10/244,696

(22) Filed: Sep. 16, 2002

Related U.S. Application Data
(60) Provisional application No. 60/322,791, filed on Sep. 17, 2001.

(51) Int. Cl. C12Q 1/70; C12Q 1/42; C12P 19/34

(52) U.S. Cl. 435/5; 435/91.2; 435/21

(57) ABSTRACT

The present invention concerns the use of methods and compositions for diagnosis, prognosis, and treatment of HIV infection and AIDS using thymidine phosphorylase as an indicator.

The present application claims priority to co-pending U.S. Patent Application Serial No. 60/322,791, filed on Sep. 17, 2001. The entire text of the above-referenced disclosure is specifically incorporated herein by reference without disclaimer.

The government may own rights in the present invention pursuant to grant number AI43244 and AI38530 from the National Institutes of Health and the National Institute of Allergy and Infectious Diseases.

### BACKGROUND OF THE INVENTION

1. Field of the Invention

The present invention relates generally to the fields of virology and immunology. More particularly, it concerns diagnostic, prognostic, and therapeutic methods and compositions for evaluating HIV infection, AIDS progression and AIDS disease management. It involves the detection of thymidine phosphorylase (TP) (also known as platelet-derived endothelial growth factor, or PDECGF) in patients infected with HIV or suspected of being infected with HIV.

2. Description of Related Art

Scientists have observed that a gradual depletion of CD4⁺ T cells from the blood characterizes AIDS and correlates with a patient's declining immunocompetency. However, the mechanism by which CD4+ T cell are lost is not fully understood. Studies have shown that there is not a significant increase of dying cells in the blood of HIV+ patients, in comparison to uninfected subjects (Groux et al., 1992; Gougeon et al., 1992), but depletion is very gradual, taking an average of 10 years, and the extent of killing may be too small to notice grossly. However, increased frequencies of dying cells are found in lymph nodes of HIV patients (Janossy et al., 1985; Muro-Cacho et al., 1995). These cells have been shown to be undergoing apoptosis and are usually not productively infected (i.e., they are not producing virus and have been termed “bystander” cells) (Finkel et al., 1995) In fact, the frequencies of infected cells making significant amounts of virus at any given time in the blood and lymph nodes of HIV+ patients are very low (approximately 1 in 100,000 cells on average) (Embretson et al., 1993). Thus, direct killing of CD4 T-cells by HIV replication could not account for much CD4 T-cell depletion. Furthermore, several studies demonstrated that as CD4 T-cell numbers decrease in the blood in early phases of the disease, the CD4 T-cells in lymph nodes do not disappear, and often increase in number for a period of time (Janossy et al., 1985; Mangkornkanok-Mark et al., 1985). Accordingly, the CD4/CD8 ratios in lymph nodes do not invert until very late in the disease (Mangkornkanok-Mark et al., 1985), in contrast to the blood. These data cannot be easily reconciled with a simple view that the disappearance of CD4 lymphocytes observed on a daily basis in the blood occurs similarly throughout the lymphoid tissues, as conjectured in certain mathematical models (Perelson et al., 1996). Using that premise, those studies speculated that a very large number of CD4 lymphocytes (109) are eliminated per day in HIV+ individuals. Furthermore, a compensatory increase in production of new CD4 lymphocytes had to be speculated, otherwise depletion of CD4 lymphocytes would occur very rapidly and AIDS would occur within weeks. In the last few years, studies examining the production of new CD4 lymphocytes have provided strong evidence that there is no major increase, but rather a slight decrease, in the production of new CD4 cells in HIV+ subjects (Hellerstein et al., 1999; Wolthers et al., 1996; Roederer et al., 1995).

Other studies have shown that HIV has an effect on resting CD4 lymphocytes, which would be the predominant cells surrounding any productively infected lymphocytes present in lymphoid tissues (since 98-99% of all lymphocytes are resting) (Janeway et al., 1996). Virus has been shown to bind and enter resting lymphocytes and to reverse transcribe partial or complete DNA proviruses. These do not integrate, however (Zack et al., 1992). This is a type of abortive infection, with the unintegrated viral DNA having a reported half-life of 6 hrs to about a week (Zack et al., 1992; Spina et al., 1995). If this abortively infected cell is activated by antigen into the cell cycle within this period, the virus can complete its replication cycle and produce progeny virions. The cell is then productively infected. The ratio of the frequencies of productively infected cells (˜7 per 10⁶) in both blood and lymph nodes to abortively infected cells (approximately 7500 per 10⁶ cells) (Chun et al., 1997) shows that greater than 99% of all HIV-infected cells in the body of an infected individual are abortively infected resting lymphocytes. This should be expected, since most CD4 lymphocytes are resting (Janeway et al., 1996). The binding of HIV signals these cells, and it has been shown that this results in up-regulation of L-selectin, the receptor for homing to lymph nodes. This receptor stays elevated for around 3 days, and these cells display enhanced binding (˜12-fold increase) to high endothelial venules in sections of lymph nodes and display enhanced homing when injected into the blood of SCID mice (Wang et al., 1997). During the homing process, a large number (40-50%) of these cells are induced into apoptosis after they enter the lymph node, which appears to be due to secondary signaling through any of several homing receptors (minimally L-selectin, CD44, CD11a) (Wang et al., 1999). Thus, a scenario has arisen from these studies which depicts resting CD4 lymphocytes in lymphoid tissues coming into contact with HIV virions, productively-infected cells, or HIV-coated follicular dendritic cells, resulting in induction of a partially activated phenotype, including upregulation of L-selectin and Fas. This is maintained for a number of days. Because of normal lymph node/blood circulation in which most lymphocytes in lymphoid tissues migrate back to the blood within two days (Ford et al., 1969), many of these cells will end up back in the blood at the time of maximally induced expression of L-selectin. These cells would then home very rapidly back to lymph nodes. Following transendothelial migration and entry into the lymph nodes, approximately half of them would be induced into apoptosis, and they do not produce HIV.

This scenario can explain many important observations in HIV+ patients: 1) as CD4 lymphocytes disappear in the blood, their numbers do not drop, and the CD4/CD8 ratios do not invert, in lymph nodes until late in disease; 2) there is no increased frequency of dying cells in the blood, but there is in lymph nodes; 3) cells that are dying in lymph nodes are not making HIV, and they are dying by apoptosis; 4) the early increase of CD4 cells in the blood following HAART treatment appears to be due to redistribution from tissues and this would be expected if the disappearance of blood CD4 cells is mainly due to HIV-induced enhanced homing (Bucy et al., 1999); and 5) steroids, which are known to down-regulate L-selectin and retard lymph node homing, have been shown to retard or stop the disappearance of CD4 cells in the blood of HIV+ patients (Sackstein et al., 1995; Andrieu et al., 1995).

More direct evidence is now needed to show whether this scenario actually occurs in HIV+ patients. Furthermore, information about this process could lead to improvements in diagnosis and prognosis for HIV-infected patients. Currently, diagnosis of HIV infection is done by an antibody test or a viral load test. Prognosis is evaluated using the viral load test and absolute CD4 cell counts, with the latter viewed as the better predictor. However, the CD4 count, at any given time, does not necessarily indicate the rate at which progression will occur. Thus, improved assays for the prognosis of HIV-infected patients are needed.

Furthermore, some patients are resistant to thymidine analog antiviral medications to varying degrees. Presently, there is no way of evaluating whether a patient is resistant to such therapeutics. Information that a patient will be resistant to the analogs can lead to better treatment options to account for the resistance.

### SUMMARY OF THE INVENTION

The present invention is based on the observation that cells exposed to the human immunodeficiency virus express higher levels of thymidine phosphorylase than cells not exposed to the virus. The present invention concerns compositions and methods for diagnosing, prognosing, and treating HIV and the disease it causes, AIDS (also referred to as HIV disease).

In some embodiments, the invention concerns methods of evaluating AIDS progression in a patient infected with HIV or suspected of being infected with HIV. A patient infected with HIV may eventually develop AIDS, but persons differ in the rate at which they develop the disease, the rate at which the diseases progresses (gets worse) and the severity of that progression. While the invention focuses on humans possibly infected with HIV, it also concerns other mammals capable of infection by a virus tantamount to HIV. For example, the invention concerns monkeys infected with SIV, and thus in any embodiment involving a patient, the patient may be a mammal, such as a monkey, chimpanzee, or gorilla. Similarly, SIV may be the virus infecting or suspected of infecting an animal.

Methods for evaluating or predicting AIDS progression in a patient infected with HIV or suspected of being infected with HIV include the following steps: a) obtaining a sample from a patient known to be infected with HIV or suspected of being infected; and b) assaying the sample for an elevated level of thymidine phosphorylase (TP). An “elevated level” refers to a level that is higher than the average level in CD4+ T cells (also referred to as “CD4 T cells” herein) not exposed to HIV. In some embodiments, an elevated level is at least or greater than 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 25, 30, 35, 40, 45, 50 or more times greater than the level in a CD4+ T cell not exposed to HIV (uninfected cell), or in a sample containing CD4+ T cells not exposed to HIV (uninfected sample). In further embodiments, an elevated level is at least a two-fold increase in expression of TP mRNA as compared to the expression level of TP mRNA in normal, uninfected cells, as determined using a microarray gene chip. In still further embodiments, the level of thymidine phosphorylase in a CD4+ T cell not exposed to HIV is undetectable or the level is equal to or less than 2% of the CD4 T cells being positive for TP in uninfected cells, if determined by FACS, under conditions as described in Example 3. By FACS, an elevated level of TP means that 4% or more CD4 T cells are positive for TP, in contrast to normal or uninfected cells. In some embodiments, an “increased level” refers to a level that is increased with respect to the level of that same patient, but at a previous time; thus, a patient may have a first TP assay performed and a later TP assay on him may show an increased level of TP relative to the earlier assay result.

It is contemplated that the sample may be any biological material obtained from a patient. In some embodiments, the sample is a blood sample, peripheral blood mononuclear cells (PBMCs) isolated from blood, saliva, cerebrospinal fluid (CSF), or any fluid or tissue sample that contains lymphocytes.

In the context of the present invention, “assaying” refers to evaluating, measuring, or testing the sample to quantitate or qualify it for an amount of thymidine phosphorylase. It is contemplated that the amount of thymidine phosphorylase may be assayed by measuring the amount of thymidine phosphorylase protein, transcript, or activity.

In some embodiments, the level of thymidine phosphorylase is assayed using an antibody directed against a thymidine phosphorylase epitope. The phrase “directed against” refers to a specific binding between the antibody and thymidine phosphorylase or the recognition by the antibody of an epitope on thymidine phosphorylase. The antibody may be employed in an ELISA assay performed on the sample. Alternatively, the level of thymidine phosphorylase is assayed immunohistochemically in some embodiments of the invention. It is contemplated that the thymidine phosphorylase antibody (anti-thymidine phosphorylase or anti-TP) may be labeled to allow its detection. It may be labeled with a radioisotope, with a colorimetric label, or with an enzymatic label such as horseradish peroxidase or potato acid phosphatase. In further embodiments, the level of TP is measured using flow cytometry.

In additional embodiments of the invention, the level of thymidine phosphorylase is assayed by measuring the level of mRNA molecules (also termed “transcripts”) encoding thymidine phosphorylase. The level of thymidine phosphorylase transcripts is measured, in some embodiments, by amplifying the transcripts.

In further embodiments, the level of thymidine phosphorylase is assayed using mass spectrometry.

The level of thymidine phosphorylase may also assayed, according to some embodiments, by measuring thymidine phosphorylase activity. This can be accomplished by the amount of substrate conversion using a thymidine phosphorylase substrate. Alternatively, binding activity between TP and a compound, such as a peptide or polypeptide, may be evaluated. Substrates that can be employed include thymidine, which is converted to thymine and 2-deoxyribose-1-phosphate, 2′-deoxy-5-fluorouridine, 5-trifluoromethyl-2-deoxyuridine (F₃dThd)m, tegafur, and 5′-deoxy-5-fluorouridine. Substrate conversion can be accomplished by methods known to skilled artisans, including by measuring the amount of converted product, amount of byproducts, amount of substrate remaining after incubation with TP, incorporation or release of a label on a substrate or converted product or byproduct.

Additional embodiments include methods of determining or evaluating whether a patient is infected with HIV or has developed AIDS. Such methods can be done by at least the following steps: a) obtaining a sample from a patient suspected of being infected with HIV; b) assaying the sample for an elevated level of thymidine phosphorylase. A patient suspected of being infected with HIV includes any patient who may be fall into any high risk group for HIV or a patient who exhibits signs consistent with infection with HIV or AIDS. Alternatively, the patient may be any person who wishes to know whether he or she is infected with HIV. It is contemplated that the patient being tested is not known to have cancer. Furthermore, it is contemplated that any embodiments discussed herein with respect to one method, may also be applied with respect to any other method. For example, methods of evaluating for HIV infection can involve embodiments contemplated for methods of evaluating AIDS progression in a patient or methods of evaluating resistance to a thymidine analog.

The present invention also includes methods of methods of evaluating resistance to a thymidine analog AIDS drug in a patient comprising: a) obtaining a sample from a patient known to be infected with HIV; and b) assaying the sample for a level of thymidine phosphorylase, wherein a elevated level of thymidine phosphorylase is indicative of risk of resistance to the thymidine analog AIDS drug.

The invention concerns not only the effect on thymidine phosphorylase levels when a cell is contacted, exposed, or infected with HIV, but it also concerns the effect of any thymidine phosphorylase on AIDS treatment regimens that include thymidine analogs. It is contemplated that methods of treating a patient infected with HIV may include: a) obtaining a sample from a patient known to be infected with HIV; b) assaying the sample for a level of thymidine phosphorylase, wherein a elevated level of thymidine phosphorylase is indicative of risk of resistance to the thymidine analog AIDS drug; and c) administering to the patient an effective amount of an AIDS drug after considering the risk of resistance to the thymidine analog AIDS drug. Such methods allow dosages and regimens of AIDS drugs to be altered or modified based on the level of TP in the patient. The level in a particular patient may be elevated with respect to the level in uninfected persons, the level in the majority of infected persons, or the level of that same patient at an earlier time, for example, when that patient was first determined to be infected with HIV but before he/she exhibited signs or symptoms of AIDS. The dosage or frequency of administration may be raised to account for the higher level, which indicates a higher level of resistance to any thymidine analogs. It is understand that the dosage or frequency is raised with respect to the dosage or frequency to a person whose TP level was unknown or not elevated or with respect to the patient's dosage prior to determining the patient's level of TP had increased or become elevated. Thus, the present method may involve determining that a patient has an elevated level of TP (compared to uninfected persons ) or even an increased level of TP with respect to the patient himself.

In further embodiments of the invention, kits that provide tools or reagents for implementing methods of the invention are provided. In one embodiment, a kit for evaluating AIDS progression in a patient is contemplated. Such a kit comprises, in a suitable container means, an antibody directed against an epitope of human thymidine phosphorylase. In some embodiments, kits further include literature indicating a first level of thymidine phosphorylase in a particular sample from a subject not infected with HIV and a second level of thymidine phosphorylase in a particular sample from a subject infected with HIV. Such literature can be used to evaluate appropriate medicines, dosage of medicines, and frequency of medicines for the patient. In still further embodiments, kits of the invention include an ELISA kit for evaluating AIDS progression in a patient comprising, in a suitable container means, a non-reacting support coupled to an antibody directed against an epitope of human thymidine phosphorylase. The non-reacting support may be cellulose or beads (glass or plastic) or a plastic container with multiple wells or raised areas to place samples. Kits or methods of the invention may include the use of standards. Standards may be provided to allow comparisons. Standards for an “elevated level” may be provided and standards for uninfected persons and/or persons showing no symptoms of AIDS may be provided.

Aspects of the invention discussed with respect to one embodiment of the invention apply to other embodiments of the invention, and vice versa.

The use of the word “a” or “an” when used in conjunction with the term “comprising” in the claims and/or the specification may mean “one,” but it is also consistent with the meaning of“one or more,” “at least one,” and “one or more than one.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Expression of TP (Thymidine phosphorylase) in resting CD4+T lymphocytes following mock-treatment or HIV-1 binding. After purifying resting CD4+ T lymphocytes from the blood of a healthy donor, 1×10⁶ T lymphocytes were incubated with different amounts of HIV-1₂₁₃ in a 37° C. CO₂ incubator for 24 hours. Mock-treated cells were incubated with media only. Cells were collected and suspended in Cytofix/Cytoperm™ solution (BD-Pharmingen) for 10-20 min at 4° C. for intracellular staining for TP. These fixed and permeabilized cells were then incubated with goat anti-TP antibody or normal goat serum (1 μg/ml) and stained with FITC-conjugated rabbit anti-goat IgG (1:50 dilution). The expression of TP was measured by flow cytometry. The normal serum gave negative staining (superimposed curves) in both mock and HIV-exposed cells.

FIG. 2. Expression of TP in resting CD4 lymphocytes at early time points after HIV exposure. Purified resting CD4 lymphocytes were exposed to HIV for 5-10 hours at 37° C. and then the cells were immunostained for expression of thymidine phosphorylase similarly to methods described in FIG. 1.

FIG. 3. HIV-induced expression of TP in resting CD4 lymphocytes is elevated for at least 5 days. As described in FIG. 1, expression of TP in cells was evaluated for five days after HIV exposure.

FIGS. 4A-4B. The level of Thymidine phosphorylase in lymphocytes of HIV (+) patients. From the collected blood of HIV (−) (FIG. 4A) and HIV (+) (FIG. 4B) donors, PBMCs were purified by LSM density gradient separation. 1×10⁶ purified PBMCs were stained with PE-conjugated anti-CD4 monoclonal antibody for 1 hour and then thoroughly resuspended I Cytofix/Cytoperm™ solution (BD-Pharmingen) for 10-20 min at 4° C. in order to perform intracellular staining for TP. The fixed and permeabilized cells were incubated with anti-TP or normal serum (isotype control) (1 μg/ml) and stained with FITC-conjugated anti-goat IgG (1:50 dilution). The expression of TP in dual-color stained cells was measured by flow cytometry, and the percentage of CD4 cells positive for TP is illustrated.

### DESCRIPTION OF ILLUSTRATIVE EMBODIMENTS

The present invention concerns the mechanism by which the blood of HIV-infected persons is depleted of CD4⁺ T cells and the observation that thymidine phosphorylase expression correlates with the number of T cells contacted with HIV and thus the number of T cells that will be depleted. The invention involves techniques, compounds, and agents that allow depleted T cells and thymidine phosphorylase levels to be evaluated to implement diagnostic, prognostic, and therapeutic methods and compositions with respect to HIV infection and AIDS.

I. HIV and AIDS

Through December 1999, more than 730,000 cases of Acquired Immune Deficiency Syndrome (AIDS) have been reported in the United States (Center for Disease Control 1999 Surveillance Report). Worldwide, it is estimated that 34.3 million people are infected with HIV or have AIDS in 1999; that same year, 5.4 million people were infected with HIV, the etiological cause of AIDS (United Nations, Report on the global HIV/AIDS epidemic, June 2000). A number of treatments have been employed singly and in combination with one another to varying degrees of effectiveness. Some of these treatments are discussed in further detail below. The effectiveness of treatments may be improved by better diagnosis and prognosis of AIDS progression. If progression or management of the disease can be more accurately tracked, treatment can be more specifically employed to improve its efficacy. For example, a patient who appears relatively healthy according to existing tests such as a viral load test may benefit from more aggressive treatment than he would have received otherwise, if implemented based on results of methods of the invention. That is, by methods of the invention, a patient may have a more serious disease progression than the viral load test indicates or may be at risk for resistance to AIDS drugs, and therefore, a more aggressive treatment is warranted than was apparent according to other known tests. On the other hand, a viral load test may indicate AIDS is progressing quite rapidly, but in fact, it is not. In this case, the patient's regimen may be relaxed compared to what it would have been without the TP assay.

A. Current HIV/AIDS Testing

To determine whether a person has been infected with HIV, a screen is performed on a sample from a person for antibodies against the HIV virus.

Once a person is determined to be infected with HIV, other tests are employed to provide a prognosis of his condition and to evaluate the progression of AIDS.

As mentioned above, a viral load test measures the amount of HIV virus in the blood. This can be done by PCR or a branched DNA (bDNA) assay. Viral load is typically reported as number of HIV copies per milliliter of blood. The tests can give a result of “undetectable,” which is the most favorable for prognosis.

T cell counts is another measurement by which disease progression is evaluated. As HIV disease progresses, T cell counts go down in number. The test is usually reported in number of CD4+ or CD8+cells per milliliter of blood. It is generally believed that a CD4+ count in the range between 500 and 1600 is considered normal. Alternatively, a ratio of CD4+ cells to CD8+ cells can be calculated. Healthy persons generally have a ratio between 0.9 and 1.9, but as AIDS progresses, the ratio can drop significantly.

Patients with no symptoms who have less than 350 T-cells or viral load over 30,000 (bDNA) or over 55,000 (PCR) are typically offered treatment. Some clinicians delay treatment for patients with 200 to 350 T-cells and viral loads under 30,000 (bDNA) or 55,000 (PCR). Patients with no symptoms who have more than 350 T-cells and viral load below 30,000 (bDNA) or 55,000 (PCR) are usually not started on a treatment. In embodiments of the invention, a person who has an elevated level of TP may be started on a treatment regimen, as discussed below.

B. Thymidine Phosphorylase

Instead of implementing only existing T-cell tests or viral load tests, the invention includes evaluating thymidine phosphorylase in a patient either singly, or in combination with these existing tests. Thymidine phosphorylase (TP) is an enzyme that converts thymidine to thymine and 2-deoxyribose-1-phosphate. The TP protein sequence is identical to a protein known as platelet-derived endothelial cell growth factor (PD-ECGF) or endothelial cell growth factor-1 (ECGF-1) or gliostatin. The cDNA and cognate polypeptide sequence of TP can be found at GenBank Accession Number M63193.

Substrates of TP include 2′-deoxy-5-fluorouridine and 5-trifluoromethyl-2-deoxyuridine (F₃dThd)m which are inactivated by TP, and tegafur and 5′-deoxy-5-fluorouridine (into 5-FU), which are activated by TP. TP may be inhibited by 6-aminoalkyl-5-halogenuracils, such as 5-chloro-6-(2-iminopyrolidino)methyluracil. Activity, and thus amount, of TP may be evaluated by monitoring a TP substrate, the reaction catalyzed by the enzyme, a compound utilized in the reaction, or a reaction product of the reaction.

While its expression has been found to be elevated in cancer cells, no correlation has been previously reported between TP expression and HIV infection. However, the techniques in the references discussing detection and measurement of TP levels in cancer cells may be applied with respect to the present invention.

II. Proteinaceous Compositions

The present invention concerns diagnostic, prognostic, and therapeutic methods and compositions concerning infection by HIV and the development of AIDS. In certain embodiments, the present invention concerns methods and compositions comprising at least one proteinaceous molecule. The proteinaceous molecule may be used as a detection reagent for a targeted molecule, which refers to a molecule whose presence and/or amount may be assayed as part of the invention. Targeted molecules of the invention include a TP-encoding transcript; a TP protein, polypeptide or peptide; a TP reaction compound (substrate or another compound involved in a TP-catalyzed reaction); a TP reaction product (a compound resulting from a TP-catalyzed reaction); HIV transcripts, proteins, polypeptides, or peptides,; or protein, polypeptide, or peptide that allows T cells to be identified, characterized, quantitated, or isolated. In some embodiments a proteinaceous molecule is used to determine the presence of thymidine phosphorylase and/or allow the amount or activity of it to be quantified (referred to herein as “TP detection reagent”). Other proteinaceous molecules may be involved with detecting HIV infection or isolating cells to be assayed for TP levels. Such proteinaceous compounds may bind directly to targeted molecule, or such compounds may be a substrate for the targeted molecule. The proteinaceous molecule may also be used, for example, in a pharmaceutical composition for the delivery of a therapeutic agent to a patient identified as being infected with HIV or having AIDS or suspected of being infected with HIV. Other proteinaceous molecules may be part of a screening assay to identify TP detection reagents. As used herein, a “proteinaceous molecule,” “proteinaceous composition,” “proteinaceous compound,” “proteinaceous chain” or “proteinaceous material” generally refers, but is not limited to, a protein of greater than about 200 amino acids or the full length endogenous sequence translated from a gene; a polypeptide of greater than about 100 amino acids; and/or a peptide of from about 3 to about 100 amino acids. All the “proteinaceous” terms described above may be used interchangeably herein.

In certain embodiments the size of the at least one proteinaceous molecule may comprise, be at least, be at most, but is not limited to, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 or greater amino molecule residues, and any range derivable therein.

As used herein, an “amino molecule” refers to any amino acid, amino acid derivative or amino acid mimic as would be known to one of ordinary skill in the art. In certain embodiments, the residues of the proteinaceous molecule are sequential, without any non-amino molecule interrupting the sequence of amino molecule residues. In other embodiments, the sequence may comprise one or more non-amino molecule moieties. In particular embodiments, the sequence of residues of the proteinaceous molecule may be interrupted by one or more non-amino molecule moieties.

In certain embodiments the proteinaceous composition comprises at least one protein, polypeptide or peptide. In further embodiments the proteinaceous composition comprises a biocompatible protein, polypeptide or peptide. As used herein, the term “biocompatible” refers to a substance which produces no significant untoward effects when applied to, or administered to, a given organism according to the methods and amounts described herein. Organisms include, but are not limited to, humans, mammals, mice, rats, monkeys, chimpanzees, gorillas, cows, horses, and pigs. Such untoward or undesirable effects are those such as significant toxicity or adverse immunological reactions. In preferred embodiments, biocompatible protein, polypeptide or peptide containing compositions will generally be mammalian proteins or peptides or synthetic proteins or peptides each essentially free from toxins, pathogens and harmful immunogens.

Proteinaceous compositions may be made by any technique known to those of skill in the art, including the expression of proteins, polypeptides or peptides through standard molecular biological techniques, the isolation of proteinaceous compounds from natural sources, or the chemical synthesis of proteinaceous materials. The nucleotide and protein, polypeptide and peptide sequences for various genes have been previously disclosed, and may be found at computerized databases known to those of ordinary skill in the art. One such database is the National Center for Biotechnology Information's Genbank and GenPept databases (http://www.ncbi.nlm.nih.gov/). The coding regions for these known genes may be amplified and/or expressed using the techniques disclosed herein or as would be know to those of ordinary skill in the art. Alternatively, various commercial preparations of proteins, polypeptides and peptides are known to those of skill in the art.

In certain embodiments a proteinaceous compound may be purified. Generally, “purified” will refer to a specific or protein, polypeptide, or peptide composition that has been subjected to fractionation to remove various other proteins, polypeptides, or peptides, and which composition substantially retains its activity, as may be assessed, for example, by the protein assays, as would be known to one of ordinary skill in the art for the specific or desired protein, polypeptide or peptide.

A. Immunological Reagents

In certain aspects of the invention, one or more antibodies against a thymidine phosphorylase polypeptide or TP-encoding nucleic acid or HIV component may be employed in methods of the invention. These antibodies may be used in various diagnostic, prognostic, or therapeutic applications, described herein below. An antibody can be used as a detection reagent to identify a targeted molecule or it can be employed to detect an amount of a substrate, for example, to indirectly provide a measurement of a targeted molecule. An antibody can also be used to determine whether a patient is infected with HIV or to evaluate the progression of AIDS generally. Such antibodies may be generated, or they may be obtained; for example, P-GF.44C is commercially available though Lab Vision, and it is a monoclonal antibody that recognizes human, rat, and mouse TP. The antibody 654-1 is a mouse antibody that recognizes human TP (Nishida et al., 1996). Mouse monoclonal antibody MoAb 104B, MoAb 232-2 and MoAb 654-1, which recognizes human dThdPase, can be obtained from Nippon Roche Co. Ltd., Tokyo, Japan. Other such antibodies are available and are contemplated for use with methods and compositions of the invention. Alternatively, an antibody may be created or produced using methods known to those of skill in the art.

As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

The term “antibody” is used to refer to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art. Means for preparing and characterizing antibodies are also well known in the art (See, e.g., Antibodies: A Laboratory Manual, Cold Spring Harbor Laboratory, 1988; incorporated herein by reference).

Monoclonal antibodies (MAbs) are recognized to have certain advantages, e.g., reproducibility and large-scale production, and their use is generally preferred. The invention thus provides monoclonal antibodies of the human, murine, monkey, rat, hamster, rabbit and even chicken origin. Due to the ease of preparation and ready availability of reagents, murine monoclonal antibodies will often be preferred.

However, “humanized” antibodies are also contemplated, as are chimeric antibodies from mouse, rat, or other species, bearing human constant and/or variable region domains, bispecific antibodies, recombinant and engineered antibodies and fragments thereof. Methods for the development of antibodies that are “custom-tailored” to the patient's dental disease are likewise known and such custom-tailored antibodies are also contemplated.

A wide range of animal species can be used for the production of antisera. Typically the animal used for production of antisera is a rabbit, a mouse, a rat, a hamster, a guinea pig or a goat. The choice of animal may be decided upon the ease of manipulation, costs or the desired amount of sera, as would be known to one of skill in the art.

Monoclonal antibodies are obtained from a population of substantially homogeneous antibodies, i.e., the individual antibodies comprising the population are identical except for possible naturally-occurring mutations that may be present in minor amounts. Thus, the modifier “monoclonal” indicates the character of the antibody as not being a mixture of discrete antibodies.

For example, the monoclonal antibodies of the invention may be made using the hybridoma method first described by Kohler and Milstein (1975), or may be made by recombinant DNA methods (Cabilly et al., U.S. Pat. No. 4,816,567).

MAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified protein, polypeptide, peptide or domain, be it a wild-type or mutant composition. The immunizing composition is administered in a manner effective to stimulate antibody producing cells.

It is also contemplated that a molecular cloning approach may be used to generate monoclonals. In one embodiment, combinatorial immunoglobulin phagemid libraries are prepared from RNA isolated from the spleen of the immunized animal, and phagemids expressing appropriate antibodies are selected by panning using cells expressing the antigen and control cells. The advantages of this approach over conventional hybridoma techniques are that approximately 10⁴ times as many antibodies can be produced and screened in a single round, and that new specificities are generated by H and L chain combination which further increases the chance of finding appropriate antibodies. In another example, LEEs or CEEs can be used to produce antigens in vitro with a cell free system. These can be used as targets for scanning single chain antibody libraries. This would enable many different antibodies to be identified very quickly without the use of animals.

Alternatively, monoclonal antibody fragments encompassed by the present invention can be synthesized using an automated peptide synthesizer, or by expression of full-length gene or of gene fragments in E. coli.

1. Antibody Conjugates

The present invention further provides antibodies to TP transcribed messages and translated proteins, polypeptides and peptides, generally of the monoclonal type, that are linked to at least one agent to form an antibody conjugate. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules which have been attached to antibodies include toxins, anti-tumor agents, therapeutic enzymes, radio-labeled nucleotides, antiviral agents, chelating agents, cytokines, growth factors, and oligo- or poly-nucleotides. By contrast, a reporter molecule is defined as any moiety which may be detected using an assay. Non-limiting examples of reporter molecules which have been conjugated to antibodies include enzymes, radiolabels, haptens, fluorescent labels, phosphorescent molecules, chemiluminescent molecules, chromophores, luminescent molecules, photoaffinity molecules, colored particles or ligands, such as biotin.

Chimeric or hybrid antibodies may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate.

Any method known in the art for separately conjugating the antibody to the detectable moiety may be employed, including those methods described by David et al. (1974); Pain et al. (1981); and Nygren (1982).

Any antibody of sufficient selectivity, specificity or affinity may be employed as the basis for an antibody conjugate. Such properties may be evaluated using conventional immunological screening methodology known to those of skill in the art. Sites for binding to biological active molecules in the antibody molecule, in addition to the canonical antigen binding sites, include sites that reside in the variable domain that can bind pathogens, B-cell superantigens, the T cell co-receptor CD4 and the HIV-1 envelope (Sasso et al., 1989; Shorki et al., 1991; Silvermann et al., 1995; Cleary et al., 1994; Lenert et al., 1990; Berberian et al., 1993; Kreier et al., 1991). In addition, the variable domain is involved in antibody self-binding (Kang et al., 1988), and contains epitopes (idiotopes) recognized by anti-antibodies (Kohler et al., 1989).

Certain examples of antibody conjugates are those conjugates in which the antibody is linked to a detectable label. “Detectable labels” are compounds and/or elements that can be detected due to their specific functional properties, and/or chemical characteristics, the use of which allows the antibody to which they are attached to be detected, and/or further quantified if desired. Another such example is the formation of a conjugate comprising an antibody linked to a cytotoxic or anti-cellular agent, and may be termed “immunotoxins”.

Antibody conjugates are generally preferred for use as diagnostic agents. Antibody diagnostics generally fall within two classes, those for use in in vitro diagnostics, such as in a variety of immunoassays, and/or those for use in vivo diagnostic protocols, generally known as “antibody-directed imaging”.

Many appropriate imaging agents are known in the art, as are methods for their attachment to antibodies (see, for e.g., U.S. Pat. Nos. 5,021,236; 4,938,948; and 4,472,509, each incorporated herein by reference). The imaging moieties used can be paramagnetic ions; radioactive isotopes; fluorochromes; NMR-detectable substances; X-ray imaging.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and especially bismuth (III).

In the case of radioactive isotopes for therapeutic and/or diagnostic application, one might mention astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m} and/or yttrium⁹⁰. ¹²⁵I is often being preferred for use in certain embodiments, and technicium^{99m} and/or indium¹¹¹ are also often preferred due to their low energy and suitability for long range detection. Radioactively labeled monoclonal antibodies of the present invention may be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies according to the invention may be labeled with technetium^{99m} by ligand exchange process, for example, by reducing pertechnate with stannous solution, chelating the reduced technetium onto a Sephadex column and applying the antibody to this column. Alternatively, direct labeling techniques may be used, e.g., by incubating pertechnate, a reducing agent such as SNCl₂, a buffer solution such as sodium-potassium phthalate solution, and the antibody. Intermediary functional groups which are often used to bind radioisotopes which exist as metallic ions to antibody are diethylenetriaminepentaacetic acid (DTPA) or ethylene diaminetetracetic acid (EDTA).

Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

Another type of antibody conjugates contemplated in the present invention are those intended primarily for use in vitro, where the antibody is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase or glucose oxidase. Preferred secondary binding ligands are biotin and/or avidin and streptavidin compounds. The use of such labels is well known to those of skill in the art and are described, for example, in U.S. Pat. Nos. 3,817,837; 3,850,752; 3,939,350; 3,996,345; 4,277,437; 4,275,149 and 4,366,241; each incorporated herein by reference.

Yet another known method of site-specific attachment of molecules to antibodies comprises the reaction of antibodies with hapten-based affinity labels. Essentially, hapten-based affinity labels react with amino acids in the antigen binding site, thereby destroying this site and blocking specific antigen reaction. However, this may not be advantageous since it results in loss of antigen binding by the antibody conjugate.

Molecules containing azido groups may also be used to form covalent bonds to proteins through reactive nitrene intermediates that are generated by low intensity ultraviolet light (Potter & Haley, 1983). In particular, 2- and 8-azido analogues of purine nucleotides have been used as site-directed photoprobes to identify nucleotide binding proteins in crude cell extracts (Owens & Haley, 1987; Atherton et al., 1985). The 2- and 8-azido nucleotides have also been used to map nucleotide binding domains of purified proteins (Khatoon et al., 1989; King et al, 1989; and Dholakia et al, 1989) and may be used as antibody binding agents.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3α-6α-diphenylglycouril-3 attached to the antibody (U.S. Pat. Nos. 4,472,509 and 4,938,948, each incorporated herein by reference). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl)propionate.

In other embodiments, derivatization of immunoglobulins by selectively introducing sulfhydryl groups in the Fc region of an immunoglobulin, using reaction conditions that do not alter the antibody combining site are contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fc region have also been disclosed in the literature (O'Shannessy et al., 1987). This approach has been reported to produce diagnostically and therapeutically promising antibodies which are currently in clinical evaluation.

B. Polyclonal Antibodies

Polyclonal antibodies are useful in the present invention regarding multiple embodiments for use as detection reagents. Polyclonal antibodies to the TP, TP substrates, or HIV polypeptides generally are raised in animals by multiple subcutaneous (sc) or intraperitoneal (ip) injections of the chimeric polypeptide and an adjuvant. It may be useful to conjugate the chimeric polypeptides or a fragment containing the target amino acid sequence to a protein that is immunogenic in the species to be immunized, e.g. keyhole limpet hemocyanin, serum albumin, bovine thyroglobulin, or soybean trypsin inhibitor using a bifunctional or derivatizing agent, for example maleimidobenzoyl sulfosuccinimide ester (conjugation through cysteine residues), N-hydroxysuccinimide (through lysine residues), glytaraldehyde, succinic anhydride, SOCl₂, or R¹N═C═NR, where R and R¹ are different alkyl groups.

Animals are immunized against the immunogenic conjugates or derivatives by combining 1 mg or 1 μg of conjugate (for rabbits or mice, respectively) with 3 volumes of Freud's complete adjuvant and injecting the solution intradermally at multiple sites. One month later the animals are boosted with 1/5 to 1/10 the original amount of conjugate in Freud's complete adjuvant by subcutaneous injection at multiple sites. 7 to 14 days later the animals are bled and the serum is assayed for anti-chimeric polypeptides antibody titer. Animals are boosted until the titer plateaus. Preferably, the animal boosted with the conjugate of the same chimeric polypeptides, but conjugated to a different protein and/or through a different cross-linking reagent. Conjugates also can be made in recombinant cell culture as protein fusions. Also, aggregating agents such as alum are used to enhance the immune response.

C. Immunodetection Methods

As discussed, in some embodiments, the present invention concerns immunodetection methods for binding, purifying, removing, quantifying and/or otherwise detecting biological components such as antigenic regions on polypeptides and peptides, particularly TP. The immunodetection methods of the present invention can be used to identify antigenic regions of a peptide, polypeptide, or protein that has prognostic or diagnostic implications, particularly with respect to HIV infection and AIDS. HIV antibodies for detection purposes are well known to those of skill in the art. See U.S. Pat. Nos. 6,074,646 and 5,587,285, specifically incorporated by reference herein.

Immunodetection methods include enzyme linked immunosorbent assay (ELISA), radioimmunoassay (RIA), immunoradiometric assay, fluoroimmunoassay, chemiluminescent assay, bioluminescent assay, and Western blot, though several others are well known to those of ordinary skill. The steps of various useful immunodetection methods have been described in the scientific literature, such as, e.g., Doolittle M H and Ben-Zeev O, 1999; Gulbis B et al., 1993; De Jager R et al., 1993; and Nakamura et al., 1987, each incorporated herein by reference.

In general, the immunobinding methods include obtaining a sample suspected of containing a protein, polypeptide and/or peptide, and contacting the sample with a first antibody, monoclonal or polyclonal, in accordance with the present invention, as the case may be, under conditions effective to allow the formation of immunocomplexes.

These methods include methods for purifying a protein, polypeptide and/or peptide from organelle, cell, tissue or organism's samples. In these instances, the antibody removes the antigenic protein, polypeptide and/or peptide component from a sample. The antibody will preferably be linked to a solid support, such as in the form of a column matrix, and the sample suspected of containing the protein, polypeptide and/or peptide antigenic component will be applied to the immobilized antibody. The unwanted components will be washed from the column, leaving the antigen immunocomplexed to the immobilized antibody to be eluted.

The immunobinding methods also include methods for detecting and quantifying the amount of an antigen component in a sample and the detection and quantification of any immune complexes formed during the binding process. Here, one would obtain a sample suspected of containing an antigen or antigenic domain, and contact the sample with an antibody against the antigen or antigenic domain, and then detect and quantify the amount of immune complexes formed under the specific conditions.

In terms of antigen detection, the biological sample analyzed may be any sample that is suspected of containing an antigen or antigenic domain, such as, for example, a tissue section or specimen, a homogenized tissue extract, a cell, an organelle, separated and/or purified forms of any of the above antigen-containing compositions, or even any biological fluid that comes into contact with the cell or tissue, including blood and/or serum.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to, any antigens present. After this time, the sample-antibody composition, such as a tissue section, ELISA plate, dot blot or western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. U.S. Patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody and/or a biotin/avidin ligand binding arrangement, as is known in the art.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the antibody is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection designed by Charles Cantor uses two different antibodies. A first step biotinylated, monoclonal or polyclonal antibody is used to detect the target antigen(s), and a second step antibody is then used to detect the biotin attached to the complexed biotin. In that method the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

1. ELISAs

As detailed above, immunoassays, in their most simple and/or direct sense, are binding assays. Certain preferred immunoassays are the various types of enzyme linked immunosorbent assays (ELISAs) and/or radioimmunoassays (RIA) known in the art. Immunohistochemical detection using tissue sections is also particularly useful. However, it will be readily appreciated that detection is not limited to such techniques, and/or western blotting, dot blotting, FACS analyses, and/or the like may also be used.

In one exemplary ELISA, antibodies are immobilized onto a selected surface exhibiting protein affinity, such as a well in a polystyrene microtiter plate. Then, a test composition suspected of containing the antigen, such as a clinical sample, is added to the wells. After binding and/or washing to remove non-specifically bound immune complexes, the bound antigen may be detected. Detection is generally achieved by the addition of another antibody that is linked to a detectable label. This type of ELISA is a simple “sandwich ELISA.” Detection may also be achieved by the addition of a second antibody, followed by the addition of a third antibody that has binding affinity for the second antibody, with the third antibody being linked to a detectable label.

In another exemplary ELISA, the samples suspected of containing the antigen are immobilized onto the well surface and/or then contacted with antibodies. After binding and/or washing to remove non-specifically bound immune complexes, the bound anti-antibodies are detected. Where the initial antibodies are linked to a detectable label, the immune complexes may be detected directly. Again, the immune complexes may be detected using a second antibody that has binding affinity for the first antibody, with the second antibody being linked to a detectable label.

Another ELISA in which the antigens are immobilized, involves the use of antibody competition in the detection. In this ELISA, labeled antibodies against an antigen are added to the wells, allowed to bind, and/or detected by means of their label. The amount of an antigen in an unknown sample is then determined by mixing the sample with the labeled antibodies against the antigen during incubation with coated wells. The presence of an antigen in the sample acts to reduce the amount of antibody against the antigen available for binding to the well and thus reduces the ultimate signal. This is also appropriate for detecting antibodies against an antigen in an unknown sample, where the unlabeled antibodies bind to the antigen-coated wells and also reduces the amount of antigen available to bind the labeled antibodies.

Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

Following all incubation steps in an ELISA, the contacted surface is washed so as to remove non-complexed material. An example of a washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. This may be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated, e.g., using a visible spectra spectrophotometer.

2. Immunohistochemistry

The antibodies of the present invention may also be used in conjunction with both fresh-frozen and/or formalin-fixed, paraffin-embedded tissue blocks or isolated blood cells prepared for study by immunohistochemistry (IHC). For example, immunohistochemistry may be utilized to evaluate the number of cells contacted with HIV by measuring the amount of TP activity or amount. The method of preparing tissue blocks from these particulate specimens has been successfully used in previous ItHC studies of various prognostic factors, and/or is well known to those of skill in the art (Brown et a., 1990; Abbondanzo et al., 1990; Allred et al., 1990).

Briefly, frozen-sections may be prepared by rehydrating 50 ng of frozen “pulverized” tissue at room temperature in phosphate buffered saline (PBS) in small plastic capsules; pelleting the particles by centrifugation; resuspending them in a viscous embedding medium (OCT); inverting the capsule and/or pelleting again by centrifugation; snap-freezing in −70° C. isopentane; cutting the plastic capsule and/or removing the frozen cylinder of tissue; securing the tissue cylinder on a cryostat microtome chuck; and/or cutting 25-50 serial sections.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections.

3. FACS

Fluorescence-activated cell sorting or cytometry (FACS) can be used to detect TP in blood cells in HIV-infected patients. Such techniques are well known to those of skill in the art. Generally, this would entail isolation of the mononuclear cells or lysis of the red blood cells in a blood sample, and then fixing and permeabilizing the cells for immunostaining for TP. The cells would be analyzed on a flow cytometer or FACS, and the percent of cells that are positive for TP can be quantitated.

III. Nucleic Acid Compositions

Certain embodiments of the present invention involve the detection of a nucleic acid or the use of a nucleic acid to express proteins used in aspects of the invention. TP expression may be evaluated by measuring the amount of TP transcript (mRNA) in a cell using a variety of techniques known to those of ordinary skill in the art and described herein. Embodiments of the invention also involve the creation and use of recombinant host cells through the application of DNA technology, that express one or more antibodies against TP. Alternatively, a nucleic acid composition may be a substrate of TP to measure its activity.

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompass the terms “oligonucleotide” and “polynucleotide,” each as a subgenus of the term “nucleic acid.” The term “oligonucleotide” refers to a molecule of between about 3 and about 100 nucleobases in length. The term “polynucleotide” refers to at least one molecule of greater than about 100 nucleobases in length.

These definitions generally refer to a single-stranded molecule, but in specific embodiments will also encompass an additional strand that is partially, substantially or fully complementary to the single-stranded molecule. Thus, a nucleic acid may encompass a double-stranded molecule or a triple-stranded molecule that comprises one or more complementary strand(s) or “complement(s)” of a particular sequence comprising a molecule. As used herein, a single stranded nucleic acid may be denoted by the prefix “ss,” a double stranded nucleic acid by the prefix “ds,” and a triple stranded nucleic acid by the prefix “ts.”

A. Nucleobases

As used herein a “nucleobase” refers to a heterocyclic base, such as for example a naturally occurring nucleobase (i.e., an A, T, G, C or U) found in at least one naturally occurring nucleic acid (i.e., DNA and RNA), and naturally or non-naturally occurring derivative(s) and analogs of such a nucleobase. A nucleobase generally can form one or more hydrogen bonds (“anneal” or “hybridize”) with at least one naturally occurring nucleobase in manner that may substitute for naturally occurring nucleobase pairing (e.g., the hydrogen bonding between A and T, G and C, and A and U).

“Purine” and/or “pyrimidine” nucleobase(s) encompass naturally occurring purine and/or pyrimidine nucleobases and also derivative(s) and analog(s) thereof, including but not limited to, those a purine or pyrimidine substituted by one or more of an alkyl, caboxyalkyl, amino, hydroxyl, halogen (i.e., fluoro, chloro, bromo, or iodo), thiol or alkylthiol moiety. Preferred alkyl (e.g., alkyl, caboxyalkyl, etc.) moieties comprise of from about 1, about 2, about 3, about 4, about 5, to about 6 carbon atoms. Other non-limiting examples of a purine or pyrimidine include a deazapurine, a 2,6-diaminopurine, a 5-fluorouracil, a xanthine, a hypoxanthine, a 8-bromoguanine, a 8-chloroguanine, a bromothymine, a 8-aminoguanine, a 8-hydroxyguanine, a 8-methylguanine, a 8-thioguanine, an azaguanine, a 2-aminopurine, a 5-ethylcytosine, a 5-methylcyosine, a 5-bromouracil, a 5-ethyluracil, a 5-iodouracil, a 5-chlorouracil, a 5-propyluracil, a thiouracil, a 2-methyladenine, a methylthioadenine, a N,N-diemethyladenine, an azaadenines, a 8-bromoadenine, a 8-hydroxyadenine, a 6-hydroxyaminopurine, a 6-thiopurine, a 4-(6-aminohexyl/cytosine), and the like.

A nucleobase may be comprised in a nucleoside or nucleotide, using any chemical or natural synthesis method described herein or known to one of ordinary skill in the art.

B. Nucleosides

As used herein, a “nucleoside” refers to an individual chemical unit comprising a nucleobase covalently attached to a nucleobase linker moiety. A non-limiting example of a “nucleobase linker moiety” is a sugar comprising 5-carbon atoms (i.e., a “5-carbon sugar”), including but not limited to a deoxyribose, a ribose, an arabinose, or a derivative or an analog of a 5-carbon sugar. Non-limiting examples of a derivative or an analog of a 5-carbon sugar include a 2′-fluoro-2′-deoxyribose or a carbocyclic sugar where a carbon is substituted for an oxygen atom in the sugar ring.

Different types of covalent attachment(s) of a nucleobase to a nucleobase linker moiety are known in the art. By way of non-limiting example, a nucleoside comprising a purine (i.e., A or G) or a 7-deazapurine nucleobase typically covalently attaches the 9 position of a purine or a 7-deazapurine to the 1′-position of a 5-carbon sugar. In another non-limiting example, a nucleoside comprising a pyrimidine nucleobase (i.e., C, T or U) typically covalently attaches a 1 position of a pyrimidine to a 1′-position of a 5-carbon sugar (Kornberg and Baker, 1992).

C. Nucleotides

As used herein, a “nucleotide” refers to a nucleoside further comprising a “backbone moiety”. A backbone moiety generally covalently attaches a nucleotide to another molecule comprising a nucleotide, or to another nucleotide to form a nucleic acid. The “backbone moiety” in naturally occurring nucleotides typically comprises a phosphorus moiety, which is covalently attached to a 5-carbon sugar. The attachment of the backbone moiety typically occurs at either the 3′- or 5′-position of the 5-carbon sugar. However, other types of attachments are known in the art, particularly when a nucleotide comprises derivatives or analogs of a naturally occurring 5-carbon sugar or phosphorus moiety.

D. Nucleic Acid Segments

In certain embodiments, the nucleic acid is a nucleic acid segment. Such nucleic acid segments may be employed as primers in the context of the present invention to detect HIV sequences or TP sequences or even CD4+ or CD8+ cells. As used herein, the term “nucleic acid segment,” are smaller fragments of a nucleic acid, such as for non-limiting example, those that encode only part of the TP peptide or polypeptide sequence. Thus, a “nucleic acid segment” may comprise any part of a gene sequence, of from about 2 nucleotides to the full length of the TP peptide or polypeptide encoding region.

Various nucleic acid segments may be designed based on a particular nucleic acid sequence, and may be of any length. By assigning numeric values to a sequence, for example, the first residue is 1, the second residue is 2, etc., an algorithm defining all nucleic acid segments can be created:

n to n+y

where n is an integer from 1 to the last number of the sequence and y is the length of the nucleic acid segment minus one, where n+y does not exceed the last number of the sequence. Thus, for a 10-mer, the nucleic acid segments correspond to bases 1 to 10, 2 to 11, 3 to 12 . . . and so on. For a 15-mer, the nucleic acid segments correspond to bases 1 to 15, 2 to 16, 3 to 17 . . . and so on. For a 20-mer, the nucleic segments correspond to bases 1 to 20, 2 to 21, 3 to 22 . . . and so on. In certain embodiments, the nucleic acid segment may be a probe or primer. As used herein, a “probe” generally refers to a nucleic acid used in a detection method or composition. As used herein, a “primer” generally refers to a nucleic acid used in an extension or amplification method or composition.

In a non-limiting example, nucleic acid segments may contain at least or up to 5, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 2000, 3000, 4000, or 5000 contiguous nucleotides, such as from SEQ ID NO: 1. Nucleic acid segments may also contain up to 10,000, 20,000, 30,000, 50,000, 100,000, 250,000, 500,000, 750,000, to 1,000,000 nucleotides in length, as well as constructs of greater size, up to and including chromosomal sizes are contemplated for use in the present invention.

The present invention also concerns the isolation or creation of a recombinant construct or a recombinant host cell through the application of recombinant nucleic acid technology known to those of skill in the art or as described herein. A recombinant construct or host cell may express a TP protein, peptide or peptide, or at least one biologically functional equivalent thereof. The recombinant host cell may be a prokaryotic cell. In a more preferred embodiment, the recombinant host cell is a eukaryotic cell. As used herein, the term “engineered” or “recombinant” cell is intended to refer to a cell into which a recombinant gene, such as a gene encoding a human thymidine phosphorylase, has been introduced. Therefore, engineered cells are distinguishable from naturally occurring cells which do not contain a recombinantly introduced gene. Engineered cells are thus cells having a gene or genes introduced through the hand of man. Recombinantly introduced genes will either be in the form of a cDNA gene (i.e., they will not contain introns), a copy of a genomic gene, or will include genes positioned adjacent to a promoter not naturally associated with the particular introduced gene.

Herein certain embodiments, a “gene” refers to a nucleic acid that is transcribed. In certain aspects, the gene includes regulatory sequences involved in transcription, or message production or composition. In particular embodiments, the gene comprises transcribed sequences that encode for a protein, polypeptide or peptide. As will be understood by those in the art, this function term “gene” includes both genomic sequences, RNA or cDNA sequences or smaller engineered nucleic acid segments, including nucleic acid segments of a non-transcribed part of a gene, including but not limited to the non-transcribed promoter or enhancer regions of a gene. Smaller engineered gene nucleic acid segments may express, or may be adapted to express using nucleic acid manipulation technology, proteins, polypeptides, domains, peptides, fusion proteins, mutants and/or such like.

The nucleic acid(s) of the present invention, regardless of the length of the sequence itself, may be combined with other nucleic acid sequences, including but not limited to, promoters, enhancers, polyadenylation signals, restriction enzyme sites, multiple cloning sites, coding segments, and the like, to create one or more nucleic acid construct(s). As used herein, a “nucleic acid construct” is a nucleic acid engineered or altered by the hand of man, and generally comprises one or more nucleic acid sequences organized by the hand of man.

In a non-limiting example, one or more nucleic acid constructs may be prepared containing about 3, about 5, about 8, about 10 to about 14, or about 15, about 20, about 30, about 40, about 50, about 100, about 200, about 500, about 1,000, about 2,000, about 3,000, about 5,000, about 10,000, about 15,000, about 20,000, about 30,000, about 50,000, about 100,000, about 250,000, about 500,000, about 750,000, to about 1,000,000 nucleotides in length, as well as constructs of greater size, up to and including chromosomal sizes (including all intermediate lengths and intermediate ranges), given the advent of nucleic acids constructs such as a yeast artificial chromosome are known to those of ordinary skill in the art. It will be readily understood that “intermediate lengths” and “intermediate ranges”, as used herein, means any length or range including or between the quoted values (i.e., all integers including and between such values). Non-limiting examples of intermediate lengths include about 11, about 12, about 13, about 16, about 17, about 18, about 19, etc.; about 21, about 22, about 23, etc.; about 31, about 32, etc.; about 51, about 52, about 53, etc.; about 101, about 102, about 103, etc.; about 151, about 152, about 153, etc.; about 1,001, about 1002, etc,; about 50,001, about 50,002, etc; about 750,001, about 750,002, etc.; about 1,000,001, about 1,000,002, etc. Non-limiting examples of intermediate ranges include about 3 to about 32, about 150 to about 500,001, about 3,032 to about 7,145, about 5,000 to about 15,000, about 20,007 to about 1,000,003, etc.

The term “functionally equivalent codon” is used herein to refer to codons that encode the same amino acid, such as the six codons for arginine and serine, and also refers to codons that encode biologically equivalent amino acids.

It will also be understood that amino acid sequences or nucleic acid sequences may include additional residues, such as additional N- or C-terminal amino acids or 5′ or 3′ sequences, or various combinations thereof, and yet still be essentially as set forth in one of the sequences disclosed herein, so long as the sequence meets the criteria set forth above, including the maintenance of biological protein, polypeptide or peptide activity where expression of a proteinaceous composition is concerned. The addition of terminal sequences particularly applies to nucleic acid sequences that may, for example, include various non-coding sequences flanking either of the 5′ and/or 3′ portions of the coding region or may include various internal sequences, i.e., introns, which are known to occur within genes.

The nucleic acids of the present invention encompass biologically functional equivalent thymidine phosphorylase or anti-TP proteins, polypeptides, or peptides or lipofuscin proteins, polypeptides or polypeptides. Such sequences may arise as a consequence of codon redundancy or functional equivalency that are known to occur naturally within nucleic acid sequences or the proteins, polypeptides or peptides thus encoded. Alternatively, functionally equivalent proteins, polypeptides or peptides may be created via the application of recombinant DNA technology, in which changes in the protein, polypeptide or peptide structure may be engineered, based on considerations of the properties of the amino acids being exchanged. Changes designed by man may be introduced, for example, through the application of site-directed mutagenesis techniques as discussed herein below, e.g., to introduce improvements or alterations to the antigenicity of the protein, polypeptide or peptide, or to test mutants in order to examine TP or anti-TP protein, polypeptide or peptide activity at the molecular level.

Fusion proteins, polypeptides or peptides may be prepared, e.g., where the coding regions are aligned within the same expression unit with other proteins, polypeptides or peptides having desired functions. Non-limiting examples of such desired functions of expression sequences include purification or immunodetection purposes for the added expression sequences, e.g., proteinaceous compositions that may be purified by affinity chromatography or the enzyme labeling of coding regions, respectively.

Encompassed by the invention are nucleic acid sequences encoding relatively small peptides or fusion peptides, such as, for example, peptides of from about 3, about 4, about 5, about 6, about 7, about 8, about 9, about 10, about 11, about 12, about 13, abou 14, about 15, about 16, about 17, about 18, about 19, about 20, about 21, about 22, about 23, about 24, about 25, about 26, about 27, about 28, about 29, about 30, about 31, about 32, about 33, about 34, about 35, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, to about 100 amino acids in length, or more preferably, of from about 15 to about 30 amino acids in length.

As used herein an “organism” may be a prokaryote, eukaryote, virus and the like. As used herein the term “sequence” encompasses both the terms “nucleic acid” and “proteancecous” or “proteanaceous composition.” As used herein, the term “proteinaceous composition” encompasses the terms “protein”, “polypeptide” and “peptide.” As used herein “artificial sequence” refers to a sequence of a nucleic acid not derived from sequence naturally occurring at a genetic locus, as well as the sequence of any proteins, polypeptides or peptides encoded by such a nucleic acid. A “synthetic sequence”, refers to a nucleic acid or proteinaceous composition produced by chemical synthesis in vitro, rather than enzymatic production in vitro (i.e., an “enzymatically produced” sequence) or biological production in vivo (i.e., a “biologically produced” sequence).

E. Nucleic Acid Complements

The present invention also encompasses a nucleic acid that is complementary to the nucleic acid encoding for a TP, HIV-nucleic acid or protein, or a protein specific for a particular T-cell population. In particular embodiments the invention encompasses a nucleic acid or a nucleic acid segment complementary to the sequence set forth in SEQ ID NO: 1, which is the cDNA sequence for human TP. A nucleic acid is “complement(s)” or is “complementary” to another nucleic acid when it is capable of base-pairing with another nucleic acid according to the standard Watson-Crick, Hoogsteen or reverse Hoogsteen binding complementarity rules. As used herein “another nucleic acid” may refer to a separate molecule or a spatial separated sequence of the same molecule.

In general, it is envisioned that the probes or primers described herein will be useful as reagents in solution hybridization, as in PCR™, for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772. The relevant portions of these and other references identified in this section of the Specification are incorporated herein by reference.

As used herein, the term “complementary” or “complement(s)” also refers to a nucleic acid comprising a sequence of consecutive nucleobases or semiconsecutive nucleobases (e.g., one or more nucleobase moieties are not present in the molecule) capable of hybridizing to another nucleic acid strand or duplex even if less than all the nucleobases do not base pair with a counterpart nucleobase. In certain embodiments, a “complementary” nucleic acid comprises a sequence in which about 70%, about 71%, about 72%, about 73%, about 74%, about 75%, about 76%, about 77%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, to about 100%, and any range derivable therein, of the nucleobase sequence is capable of base-pairing with a single or double stranded nucleic acid molecule during hybridization. In certain embodiments, the term “complementary” refers to a nucleic acid that may hybridize to another nucleic acid strand or duplex in stringent conditions, as would be understood by one of ordinary skill in the art.

In certain embodiments, a “partly complementary” nucleic acid comprises a sequence that may hybridize in low stringency conditions to a single or double stranded nucleic acid, or contains a sequence in which less than about 70% of the nucleobase sequence is capable of base-pairing with a single or double stranded nucleic acid molecule during hybridization.

F. Nucleic Acid Detection

In addition to their use in directing the expression of SEQ ID NO: 2 or anti-TP antibodies, proteins, polypeptides and/or peptides, the nucleic acid sequences disclosed herein have a variety of other uses. For example, they have utility as probes or primers for embodiments involving nucleic acid hybridization, particularly those that contain all or part of SEQ ID NO:1. They also can be used for determining the activity of thymidine phosphorylase. For example, the transcript levels of TP can be measured to determine the level of TP in a sample.

As used herein, “hybridization”, “hybridizes” or “capable of hybridizing” is understood to mean the forming of a double or triple stranded molecule or a molecule with partial double or triple stranded nature. The term “anneal” as used herein is synonymous with “hybridize.” The term “hybridization”, “hybridize(s)” or “capable of hybridizing” encompasses the terms “stringent condition(s)” or “high stringency” and the terms “low stringency” or “low stringency condition(s).”

As used herein “stringent condition(s)” or “high stringency” are those conditions that allow hybridization between or within one or more nucleic acid strand(s) containing complementary sequence(s), but precludes hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating a nucleic acid, such as a gene or a nucleic acid segment thereof, or detecting at least one specific mRNA transcript or a nucleic acid segment thereof, and the like.

Stringent conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid(s), the length and nucleobase content of the target sequence(s), the charge composition of the nucleic acid(s), and to the presence or concentration of formamide, tetramethylammonium chloride or other solvent(s) in a hybridization mixture.

It is also understood that these ranges, compositions and conditions for hybridization are mentioned by way of non-limiting examples only, and that the desired stringency for a particular hybridization reaction is often determined empirically by comparison to one or more positive or negative controls. Depending on the application envisioned it is preferred to employ varying conditions of hybridization to achieve varying degrees of selectivity of a nucleic acid towards a -target sequence. In a non-limiting example, identification or isolation of a related target nucleic acid that does not hybridize to a nucleic acid under stringent conditions may be achieved by hybridization at low temperature and/or high ionic strength. Such conditions are termed “low stringency” or “low stringency conditions”, and non-limiting examples of low stringency include hybridization performed at about 0.15 M to about 0.9 M NaCl at a temperature range of about 20° C. to about 50° C. Of course, it is within the skill of one in the art to further modify the low or high stringency conditions to suite a particular application.

In addition to gel electrophoresis, separation of nucleic acids may also be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

Other methods of nucleic acid detection that may be used in the practice of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

G. Preparation of Nucleic Acids

A nucleic acid may be made by any technique known to one of ordinary skill in the art, such as for example, chemical synthesis, enzymatic production or biological production. Non-limiting examples of a synthetic nucleic acid (e.g., a synthetic oligonucleotide), include a nucleic acid made by in vitro chemically synthesis using phosphotriester, phosphite or phosphoramidite chemistry and solid phase techniques such as described in EP 266,032, incorporated herein by reference, or via deoxynucleoside H-phosphonate intermediates as described by Froehler et al., 1986 and U.S. Pat. No. 5,705,629, each incorporated herein by reference. In the methods of the present invention, one or more oligonucleotide may be used. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

A non-limiting example of an enzymatically produced nucleic acid include one produced by enzymes in amplification reactions such as PCR™ (see for example, U.S. Pat. Nos. 4,683,202 and 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al. 1989, incorporated herein by reference).

H. Purification of Nucleic Acids

A nucleic acid may be purified on polyacrylamide gels, cesium chloride centrifugation gradients, or by any other means known to one of ordinary skill in the art (see for example, Sambrook et al., 1989 and 2001, incorporated herein by reference).

In certain aspect, the present invention concerns a nucleic acid that is an isolated nucleic acid. As used herein, the term “isolated nucleic acid” refers to a nucleic acid molecule (e.g., an RNA or DNA molecule) that has been isolated free of, or is otherwise free of, the bulk of the total genomic and transcribed nucleic acids of one or more cells. In certain embodiments, “isolated nucleic acid” refers to a nucleic acid that has been isolated free of, or is otherwise free of, bulk of cellular components or in vitro reaction components such as for example, macromolecules such as lipids or proteins, small biological molecules, and the like.

I. Nucleic Acid Vectors

In some aspects of the invention, recombinant DNA technology is employed to create compositions of the invention or compositions for use with methods of the invention. For example, recombinant DNA technology may be used to create detection reagents specific for TP, such as a TP-specific antibody, nucleic acid sequences that hybridize to a TP-encoding nucleic acid, or a TP substrate. Alternatively, recombinant DNA technology may be employed to determine whether a patient is infected with HIV or whether they have developed symptoms of AIDS.

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Sambrooke et al., 2001 and Ausubel et al., 1994, both incorporated herein by reference).

The term “expression vector” refers to any type of genetic construct comprising a nucleic acid coding for a RNA capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operable linked coding sequence in a particular host cell. In addition to control sequences that govern transcription (promoters and enhancers) and translation, vectors and expression vectors may contain nucleic acid sequences that serve other functions as well that are well known to those of skill in the art, such as screenable and selectable markers, ribosome binding site, multiple cloning sites, splicing sites, poly A sequences, origins of replication, and other sequences that allow expression in different hosts.

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name Maxbac® 2.0 from Invitrogen® and Bacpack™ Baculovirus Expression System from Clontech®.

Other examples of expression systems include Stratagene®'s Complete Control™ Inducible Mammalian Expression System, which involves a synthetic ecdysone-inducible receptor, or its pET Expression System, an E. coli expression system. Another example of an inducible expression system is available from Invirrogen®, which carries the T-Rex™ (tetracycline-regulated expression) System, an inducible mammalian expression system that uses the full-length CMV promoter. Invitrogen® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

It is contemplated that the proteins, polypeptides or peptides produced by the methods of the invention may be “overexpressed”, i.e., expressed in increased levels relative to its natural expression in cells. Such overexpression may be assessed by a variety of methods, including radio-labeling and/or protein purification. However, simple and direct methods are preferred, for example, those involving SDS/PAGE and protein staining or western blotting, followed by quantitative analyses, such as densitometric scanning of the resultant gel or blot. A specific increase in the level of the recombinant protein, polypeptide or peptide in comparison to the level in natural cells is indicative of overexpression, as is a relative abundance of the specific protein, polypeptides or peptides in relation to the other proteins produced by the host cell and, e.g., visible on a gel.

In some embodiments, the expressed proteinaceous sequence forms an inclusion body in the host cell, the host cells are lysed, for example, by disruption in a cell homogenizer, washed and/or centrifuged to separate the dense inclusion bodies and cell membranes from the soluble cell components. This centrifugation can be performed under conditions whereby the dense inclusion bodies are selectively enriched by incorporation of sugars, such as sucrose, into the buffer and centrifugation at a selective speed. Inclusion bodies may be solubilized in solutions containing high concentrations of urea (e.g. 8M) or chaotropic agents such as guanidine hydrochloride in the presence of reducing agents, such as β-mercaptoethanol or DTT (dithiothreitol), and refolded into a more desirable conformation, as would be known to one of ordinary skill in the art.

The nucleotide and protein, polypeptide and peptide sequences for various genes have been previously disclosed, and may be found at computerized databases known to those of ordinary skill in the art. One such database is the National Center for Biotechnology Information's Genbank and GenPept databases (http://www.ncbi.nlm.nih.gov/). The coding regions for these known genes may be amplified and/or expressed using the techniques disclosed herein or by any technique that would be know to those of ordinary skill in the art. Additionally, peptide sequences may be synthesized by methods known to those of ordinary skill in the art, such as peptide synthesis using automated peptide synthesis machines, such as those available from Applied Biosystems (Foster City, Calif.).

IV. Therapies

In order to take advantage of the prognostic and diagnostic information obtained from the methods of the present invention, such as those involving the detection of TP, it may be desirable to combine them with therapeutic regimens for the treatment of HIV and AIDS. These regimens will involve agents effective in the treatment of AIDS or a particular disease or condition associated with AIDS. It is contemplated that a wide variety of conditions or diseases may be treated, such as microbial pathogenesis—including pneumonia, CMV infection, Staph and Streptococcus infection—in addition to hyperproliferative disorders including cancers such as sarcomas and leukemias. The treatment of AIDS, cancer, and microbial infection is specifically contemplated.

A. HIV/AIDS Therapies

Currently, there are three categories of drugs being used as HIV antiviral drugs: 1) nucleoside analog reverse transcriptase inhibitors (NUKES); 2) non-nucleoside reverse transcriptase inhibitors (NNRTIs); and 3) protease inhibitors. Other categories of drugs for the treatment of HIV and AIDS are also under development. It is contemplated that the diagnostic and prognostic methods of the invention may be implemented in conjunction with therapy against HIV and AIDS. Thus, if a person suspected of being infected with HIV or diagnosed as HIV-infected is evaluated for TP, then the results of that evaluation may affect whether to administer HIV/AIDS therapy or some other therapy that may be needed as a result of AIDS, as well as what therapy to administer. Furthermore, a patient may be evaluated for resistance to thymidine analogs, which have been used as an antiviral treatment. If a patient is determined to have a level of TP that is higher than normal, the therapy for that patient can include a higher dose of the thymidine analog than is usually given to a patient or it may not use the analog and use another antiviral therapy instead. The compounds discussed in detail below may be implemented with any of the methods described herein and in any acceptable combination.

1. Nucleoside Analog Reverse Transcriptase Inhibitors (NUKES)

Nucleoside analog reverse transcriptase inhibitors block reverse transcription by mimicking the nucleotides that are incorporated into a molecule being generated from a template and thus blocking transcription. They include Abacavir (Ziagen®) or 1592U89; AZT or Zidovudine (Retrovir®); ddI or Didanosine (Videx®); ddC or dideoxycytidine or Zalcitabine (Hivid®); d4T or Stavudine (Zerit®), 3TC or Lamivudine (Epivir®)); Zidovudine/Lamivudine (Combivir®); and Zidovudine/Lamivudine/Abacavir (Trizivir®). One of the most well known therapies is AZT, which may be given as an early treatment, when there are no symptoms of disease, or it may be given once symptoms of disease are observed, or when CD4+ T cell count is below 500 or when the patient has a viral load over 30,000.

2. Non-Nucleoside Analog Reverse Transcriptase Inhibitors (NNRTIs)

Non-nucleoside reverse transcriptase inhibitors (NNRTIs or non-nukes) bind reverse transcriptase to inhibit its activity. These compounds include Nevirapine (NVP) or BI-RG-587 (Viramune®); Delavirdine or DLV (Rescriptor®); and Efavirenz (EFV) or DMP-266 (Sustiva®).

3. Protease Inhibitors

Protease inhibitors prevent HIV protease from cutting proteins for assembly of new virus. Thus, new viral particles cannot mature. Protease inhibitors include Amprenavir (APV) or 141W94 (Agenerase®); Indinavir or IDV (Crixivan®); Lopinavir or ABT-378/r (Kaletra®); Nelfinavir or NFV (Viracept®); Ritonavir or RTV (Norvir®); and Saquinavir or SQV (Invirase®). Other protease inhibitors that are in development include BMS232632, GW433908, L-756,423, Mozenavir (DMP450), and Timpranavir (PNU-140690).

4. Other Antiviral Therapies

Other antiviral therapies that may be used as part of methods and compositions of the invention include attachment and fusion inhibitors, integrase inhibitors, zinc finger inhibitors, antisense drugs, and immune stimulators. Attachment and fusion inhibitors act by preventing the virus from attaching to a cell and breaking through the cell's membrane. Examples of these inhibitors are AMD-3100 (AnorMED), FP21399 (Fuji Pharmaceuticals), PRO 542 (Progenics Pharmaceuticals), T-20 (Pentafuside, Trimeris and Roche), SC351125 (Schering Plough) and T-1249 (Trimeris and Roche). Integrase inhibitors prevents the HIV transcribed product from integrating into the cell's genome; AR-177 (Zintevir, Aronex Pharmaceuticals) is an integrase inhibitor. Azodicarbonamide (ADA) is a zinc finger inhibitor that disrupts the zinf fingers that hold together the nucleocapsid of HIV. Another antiviral therapy is antisense drugs, including HGTV43 from Enzo Therapeutics. Finally, immune stimulators may be employed as a therapeutic regimen against HIV and HIV disease (AIDS). IL-2 (Aldesleukin®, Proleukin®), Reticulose, Multikine, Ampligen, HE2000, and HIV-1 Immunogen (Remune®) are example of immune stimulators.

B. Microbial Therapies

Opportunistic infection by a microbial pathogen accompanies the weakening of the immune system as a result of AIDS/HIV disease. Thus, as a result of a the methods of the invention, therapies against these microbial pathogens may be instituted by themselves or in combination with other therapies, such as antiviral therapies. The most common infections include Candidiasis (Thrush), a fungal infection that can occur even with fairly high T-cell count; Cytomegalovirus (CMV) a herpesviral infection that occurs when the T cell count is under 50; Herpes simplex viruses, other herpesviral infection; Mycobacterium avium complex (MAC or MAI), a bacterial infection that occurs when the T cell count is under 75; Pneumocystis carinii pneumonia (PCP), a protozoal infection that affecta patients with a T-cell range under 200; Toxoplasmosis (Toxo), a protozoal infection occurring when the T-cell range is under 100; and Tuberculosis (TB), a bacterial infection that can occur in anyone infected with HIV.

Antifungal treatments include locally administered compositions that contain clotrimazole, ketoconazole, nystatin, miconazole, terconazole, butoconazole, or amphotericin. Sytemic treatment can be administered as pills that contain ketoconazole (Nizoral), fluconazole (Diflucan), or itraconazole (Sporanox).

Herpesviral treatment or prevention includes administration of ganciclovir, foscarnet, cidofovr, fomivirsen, and/or valganciclovir. The treatment of other opportunistic viral infections may be administered to a patient diagnosed with HIV infection.

Antibiotics may also be administered to a patient after they have been diagnosed with HIV infection or AIDS/HIV disease using methods of the invention. TB may be treated with isoniazid (INH) and other well known and widely used antibiotics for TB. Other bacterial infections such as MAC may be treated with amikacin, azithromycin, ciprofloxacin, clarithromycin, clofazimine, ethambutol, rifabutin, or a combination thereof. Pneumonia or other protozoal infection may be treated with TMP/SMX, dapsone, pentamidine, or atovaquone.

C. Cancer Therapies

Cancer therapies also include a variety of combination therapies with traditional cancer therapies such as surgery and chemical- and radiation-based treatments. Chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein tansferase inhibitors, transplatinum, 5-fluorouracil, vincristin, vinblastin and methotrexate, or any analog or derivative variant of the foregoing.

Radiotherapies are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that the present invention may be used in conjunction with removal of superficial cancers, precancers, or incidental amounts of normal tissue.

Other cancer therapies are also contemplated, including immunotherapy, which generally relies on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. Generally, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

Gene therapy for the treatment cancer, as well as opportunistic infections of HIV is also contemplated. Cancer gene therapy may involve administering a tumor suppressor or an inhibitor of an oncogene.

V. Kits

All the essential materials and/or reagents required for detecting thymidine phosphorylase in a sample may be assembled together in a kit. This generally will comprise an detection reagent specific for thymidine phosphorylase. In one embodiment, the kit comprises an antibody against thymidine phosphorylase. The antibody may be labeled or the kit may contain other reagents to identify or isolate antibody that is binding to TP. In other embodiments, the kit contains a probe or primers designed to hybridize specifically to TP-encoding nucleic acids. Also included may be enzymes suitable for amplifying nucleic acids, including various polymerases (reverse transcriptase, Taq, etc.), deoxynucleotides and buffers to provide the necessary reaction mixture for amplification. Such kits may also include enzymes and other reagents suitable for detection of specific nucleic acids or amplification products. Such kits generally will comprise, in suitable means, distinct containers for each individual reagent or enzyme as well as for each probe or primer pair.

### VI. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Human Subjects

Three healthy uninfected donors, three HIV+ patients, who were not on antiviral drug treatment yet, and one chronic hepatitis-B patient were recruited. All were volunteers who were explained the study and read and signed IRB-approved consent forms. The CD4 counts and HIV loads for the HIV+ patients are listed in Table 1.

Lymphocyte Purification and Radiolabeling with ¹¹¹In

Seventy-five to one hundred ml of venous blood was drawn from the above volunteers into heparized tubes. The blood was centrifuged, and the plasma was collected and saved for later use. Peripheral blood mononuclear cells (PBMCs) were isolated from the blood by centrifugation through Lymphocyte Separation Medium (Organon, Teknika Corporation) and washed twice with Hanks balanced salt solution (HBSS). The cell pellet was resuspended in RPMI 1640 medium (Gibco) supplemented with 10% autologous plasma at 1×10⁶ cells/ml. Enriched CD4+ T lymphocytes were obtained from the PBMCs by negative panning procedures. Briefly, petri dishes were pretreated with 10 ml of affinity-purified goat anti-mouse (GAM) IgG (Sigma, St. Louis, Mo.) in HBSS (5 μg/ml) overnight at 4° C. The dishes were then rinsed with 10 ml of HBSS containing 2% autologous plasma five times and incubated for 1 hr at 4° C. with 20 ml of the same solution. The PBMCs were incubated in 100 pd of customized antibody cocktail (Stem Cell Technology, Vancouver) for 1 hr at 4° C. with constant mixing, washed twice, and then placed onto the GAM-IgG-coated plates for 3 hrs at 4° C. The antibody cocktail contained MoAbs to CD14, CD16, CD19, CD56 (all at 30μg/ml) and glycoporin A (10 μg/ml). Non-adherent cells were then collected, washed, and kept in supplemented RPMI 1640 media. An aliquot of the purified cell population was analyzed by flow cytometry and the percentage of CD4+ cells was determined (varied between 90-95% purity).

The cells were then delivered to the Department of Nuclear Medicine for ¹¹¹In labeling and injection. The saved autologous plasma was centrifuged at 2450 RPM for 20 minutes to produce platelet-poor plasma. The purified CD4 lymphocytes were resuspended in 6 ml of 0.9% saline, and the solution was drawn up gently into a syringe and dispensed back into the tube. This process was repeated until the button of CD4 cells was completely dispersed. One mCi of ¹¹¹In oxine was added drop-wise to the CD4 cells suspension, and the mixture of CD4 cells and ¹¹¹In oxine were incubated for 30 minutes at room temperature. The mixture was gently agitated 3-4 times during the incubation period and the CD4 cells/¹¹¹In oxine mixture was brought up to a volume of 15 ml with appropriate volume of platelet-poor plasma. The suspension was centrifuged at 1400 RPM for 5 minutes. After spinning, the supernatant was withdrawn from the tube using a syringe and spinal needle without disturbing the CD4 cell pellet. The supernatant which contain the unincorporated ¹¹¹In oxine was discarded. Eight ml of platelet-poor plasma was added to the CD4 cell platelet and the cells were resuspended. An aliquot was analyzed on a gamma counter to determine the efficiency of labeling. Five hundred μCi of ¹¹¹In labeled-CD4 cells were dispensed into a syringe and injected intravenously (anti-cubital fosa) into the original donor.

Camera and Scanning

For the total body scintophotos, the subjects were imaged ˜1, 3 and 24 hrs. postinjection with a dual-head gamma camera (Vertex, Adac Laboratories, CA.) equipped with medium-energy general purpose collimators. Flood correction was done with Indium intrinsic floods and two 20% energy windows at 173 and 250 KeV were used. For static planar view, 600 seconds/frame were collected on the chest and pelvic area. The matrix size was 256×256×6. The scan speed for the total body was S cm/min and the matrix size was high resolution-8 deep. Total body and planer scans were interpreted visually by two experienced nuclear medicine physicians from a computer display. Images were analyzed using the Pegasys processing terminals, irregular regions of interest (ROI) in each organ were drawn, duplicated, mirrored, and positioned on the sites of interest. Average counts in the ROIs were obtained as counts/pixel.

### Example 2

### CD4 Lymphoctyes in HIV+ Individuals Migrate at Enhanced Rate to Lymph Nodes and Bone Marrow

Three uninfected volunteers and three HIV-infected individuals were recruited for the study. The HIV+ donors had been diagnosed with HIV for variable amounts of time, and none were taking anti-retroviral drug therapy. Their CD4 counts ranged from 396-594 cells/μl, and viral loads ranged from 25,427-271,552 RNA copies/ml (Table 1). After signing IRB-approved informed consent, they donated between 75-100 ml of blood. Mononuclear cells were isolated from the blood, enriched for CD4 T-cells by negative-selection panning (90-95% pure), and these cells were then labeled in vitro with ¹¹¹In (1 mCi total). Following rinsing, 0.5 mCi of labeled cells were infused intravenously back into the original donors, so each donor received the same amount of radioactivity. The subjects were then scanned with a gamma camera 1, 3, and 24 hrs later, and localization of the CD4 cells was determined and quantitated.

TABLE 1
CD4 cell counts and viral loads of HIV+ volunteers

| Subject | CD4 (cells/μ1 blood) | HIV Load (RNA copies/ml plasma) |
|---|---|---|
| 1 | 503 | 27,694 |
| 2 | 396 | 271,552 |
| 3 | 594 | 25,427 |

Whole-body scinto-photos were taken of uninfected and HIV+ volunteers at various time points post-infusion. An obvious finding is the increased intensity at 1 and 3 hrs of labeled CD4 T-cells in the vertebral and iliac bone marrow and cervical lymph nodes in the HIV+patient compared to the control. This demonstrates that more CD4 T-cells migrated to these areas per unit time in the HIV+ subject compared to the uninfected subject. The two other HIV+ subjects tested displayed similar enhanced localization of CD4 T-cells in the bone, compared to the two other control subjects (Table 2). It was also obvious that most of the labeled CD4 T-cells in normal subjects migrated to bone marrow, and this was almost exclusively to vertebral and iliac marrow, and not marrow in the long bones in both types of subjects. Also, the auxiliary lymph nodes of the HIV+ patient contained more labeled cells than the control subjects at 1 and 3 hrs post-infusion. By 24 hrs, the labeled cells appeared to have distributed homogenously, and there were no significant differences between control and HIV+ subjects at that time point.

TABLE 2
Quantitation of ¹¹¹In-labeled CD4 T-cells
in various organs at 1 and 3 hrs post-infusion

| | | Lymph Nodes⁽²⁾ | |
|---|---|---|---|
| Subject | Bone⁽¹⁾ | Cervical | Axillary |
| | | 1 hr | |
| Uninfected - 1 | 1448⁽³⁾ | 1874 | 1663 |
| Uninfected - 2 | 756 | 2056 | 2358 |
| Uninfected - 3 | 1487 | 1663 | 2603 |
| (Ave. ± SD) | (1234 ± 414) | (1864 ± 197) | (2208 ± 488) |
| HIV-infected - 1 | 2385 | 3570 | 7322 |
| HIV-infected - 2 | 3173 | 2528 | 4861 |
| HIV-infected - 3 | 2265 | 3245 | 5414 |
| (Ave. ± SD) | (2608 ± 493)* | (3114 ± 533)* | (5865 +1291)* |
| HBV-infected - 1 | 971 ± 257 | 841 ± 53 | 1407 ± 209 |
| HBV-infected - 2 | 1000 ± 300 | 650 ± 38 | 1653 ± 300 |
| | | 3 hr | |
| Uninfected - 1 | 1566 | 2632 | 1796 |
| Uninfected - 2 | 2015 | 2094 | 1876 |
| Uninfected - 3 | 2357 | 1877 | 2523 |
| (Ave. ± SD) | (1979 ± 396) | (2201 ± 389) | (2051 ± 408) |
| HIV-infected - 1 | 4560 | 3930 | 2901 |
| HIV-infected - 2 | 3331 | 3683 | 3054 |
| HIV-infected - 3 | 4200 | 4095 | 4508 |
| (Ave. ± SD) | (4030 ± 631)* | (3936 ± 256)* | (3488 ± 886)* |
| HBV-infected - 1 | 1071 ± 290 | 937 ± 71 | 1480 ± 40 |
| HBV-infected - 2 | 1100 ± 350 | 780 ± 52 | 1729 ± 51 |

⁽¹⁾Counts in both vertebral and iliac bones were combined.
⁽²⁾Counts in all cervical nodes were combined; same for axillary nodes.
⁽³⁾Average radioactivity as counts per 460 pixel area per 600 seconds.
*The differences between HIV-infected and control subjects are statistically significant (p<0.01).

Higher resolution of the pelvic area for quantitation on all subjects demonstrated that there was approximately 2-fold more labeled CD4 lymphocytes in the bone marrow at 1 and 3 hrs in the HIV patients in comparison to the three controls (Table 2). High resolution of the chest and neck regions demonstrated greater CD4 T-cell localization in cervical and axillary lymph nodes in all three HIV+subjects compared to the three uninfected subjects. This was determined to be −2-fold higher (Table 2). The lungs contained the greatest percentage of labeled cells at 1 and 3 hrs post-infusion in all subjects, but that was expected since the lung would be the first major organ where the infused labeled cells will travel, and a large percentage of them will stay in the lung for the first few hrs. There was no evidence for enhanced homing of CD4 T-cells to gut tissues, CNS, or any other organs in the body.

Since HIV-infected were compared to uninfected subjects, CD4 lymphocytes were evaluated to determined whether they displayed enhanced migration in another viral (non-HIV) infection, to ascertain whether enhanced CD4 T-cell homing is a common feature of all viral infections. A patient with chronic HBV infection volunteered for this study. The results, shown in Table 2, demonstrate that CD4 lymphocytes in the blood of this HBV-infected subject migrated slightly less to bone marrow, and considerably less to cervical and auxiliary lymph nodes in comparison to control subjects. The reason for this is not clear. The subject was taking the anti-depressant Serzone, which may have some effect on the immune system (Neveu, 1999). Thus, the enhanced migration observed in HIV+ subjects appears somewhat specific for HIV infection with active viral replication, as determined by detectable virus on quantitative HIV RNA assays.

To further confirm this, the first HIV+ volunteer (#1) who had been on HAART for 3 months since his first scan was re-tested. Virus load at the initial test had been 27,694 copies/ml and at repeat testing was <400 copies/ml. Photos showed that his CD4 T-cells were now migrating at rates similar to those of uninfected subjects. His blood CD4 count had also gone up from 503-692 cells/μl. Thus the enhanced homing of CD4 lymphocytes in HIV+ patients appears to correlate with the presence of replicating HIV.

### Example 3

### Expression of Thymidine Phosphorykase in HIV Patients

To investigate the changes in RNA expression of resting CD4+ T lymphocytes by HIV-1, Affymetrix GeneChip® Expression Analysis was done. Purified resting CD4+ T lymphocytes (purity of >98%) prepared from a healthy donor by StemCell™ magnetic column technique were incubated with HIV-1₂₁₃ ((M.O.I. 0.5-1), IL-16 (10 ng/ml), or MHC II peptide (50 μM, RK1) in a 37° C. humidified 5% CO₂ incubator for 3 hrs). Then, total cytoplasmic RNA was extracted by using RNA isolation kit (Qiagen) according to the manufacturer's instructions. Isolated RNA was applied on the Human Genome U95A chips, containing 12,626 full-length genes (Affymetrix, Santa Clara, Calif.). Data were generated from hybridization intensities measured on GeneChip expression probe arrays and analyzed by GeneChip software provided by Affymetrix. In order to compare gene expression levels between two samples (Mock, HIV₂₁₃, IL-16 and MHC II peptide), Comparison Analysis was performed on data from two separate probe array experiments by determining the relative changes in abundance for each transcript. All moderate increase, moderate decrease and no change were removed. All fold changes from +2.0 to −2.0 were also removed. Based on the data, 53 probe sets were found to be changed in HIV₂₁₃ signaled cells compared to Mock control (84 genes were changed: Mock vs. IL-16, 42 genes were changed: Mock vs. MHC II, 203 genes were changed: HIV vs. IL-16, 75 genes were changed: HIV vs. MHC II, 24 genes were changed: IL-16 vs. MHC II). TP (PDECGF) was one of the molecules that have shown great change in its mRNA level when CD4 T cells were signalled with HIV (48 fold change). It was confirmed that this molecule can be highly upregulated in protein level by HIV signal, too (˜10 fold change). PDECGF level was also checked in CD4 T cell from both healthy people and HIV-positive people. Eight healthy people showed very low level of PDECGF and 3 out of 6 HIV patients showed high level of PDECGF.

Following from the gene chip data, which showed that HIV binding to CD4 cells induced upregulation of thymidine phosphorylase, a polyclonal goat antiserum to human thymidine phosphorylase (R & D System, Inc., Minneapolis, Minn.) was obtained to perform intracellular immunostaining for this protein. Since TP is expressed intracellularly, the cytoperm technique that is used for intracellular staining of cytokines was employed. This technique fixes the cells and permealizes the membranes so that antibodies can go into the cell. It may be performed as follows:

a. Collect cells ((0.5-1)×10⁶) from each well.

b. Wash cells with 2% CS-HBSS once.

c. Fix the cells using Pharmingen's Cytofix/Cytoperm™ solution (Catalog #2090KZ).

(Thoroughly resuspend cells in 100 μl (0.9×10⁶/200 μl) of Cytofix/Cytoperm™ solution for 10-20 min at 4° C.).

d. Permeabilize fixed cells by washing 2 times in 1×Perm/Wash™ buffer (Catalog # 554723).

e. Incubate for 15 minutes in 1×Perm/Wash™ buffer.

f. Pellet cells.

g. Stain for intracellular TP

1. Thoroughly resuspend cells in 50 μl of 1×Perm/Wash™ buffer containing 1 μg/ml of anti-TP at 4° C. for 30-40 minutes.

2. Wash cells 2 times with 1×Perm/Wash™ buffer and resuspend in 20 μl of 1×Pern/Wash™ buffer containing anti-goat IgG (whole molecule)-FITC conjugate.

3. Incubate for 40 min at 4° C.

4. Wash cells 2 times with 1×Perm/Wash™ buffer and resuspend in a buffer prior to FACS analysis.

h. FACS analysis

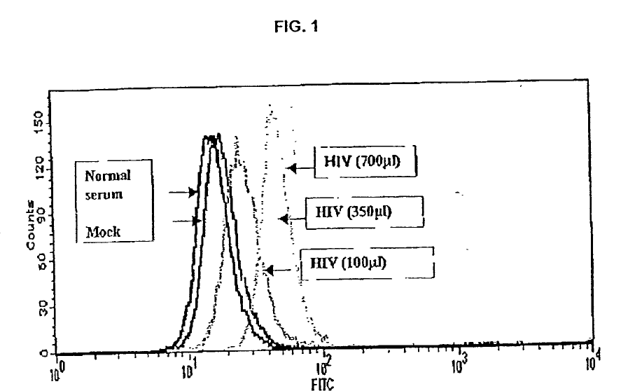

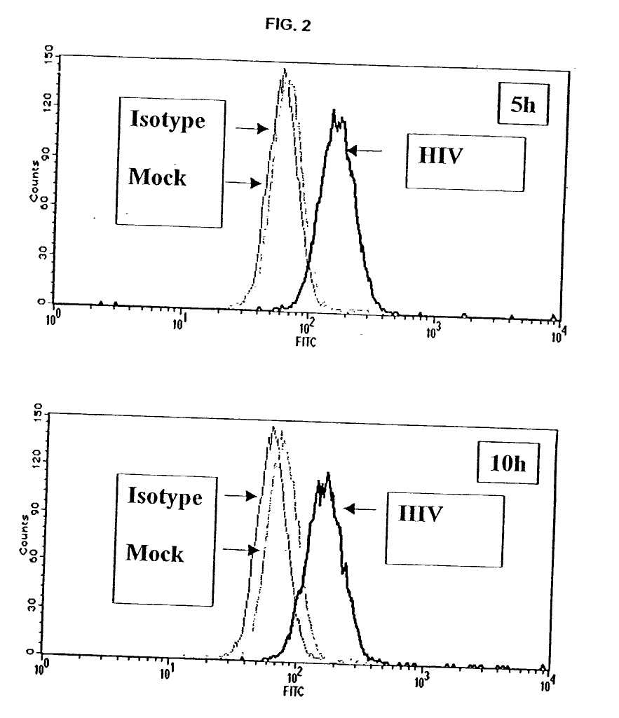

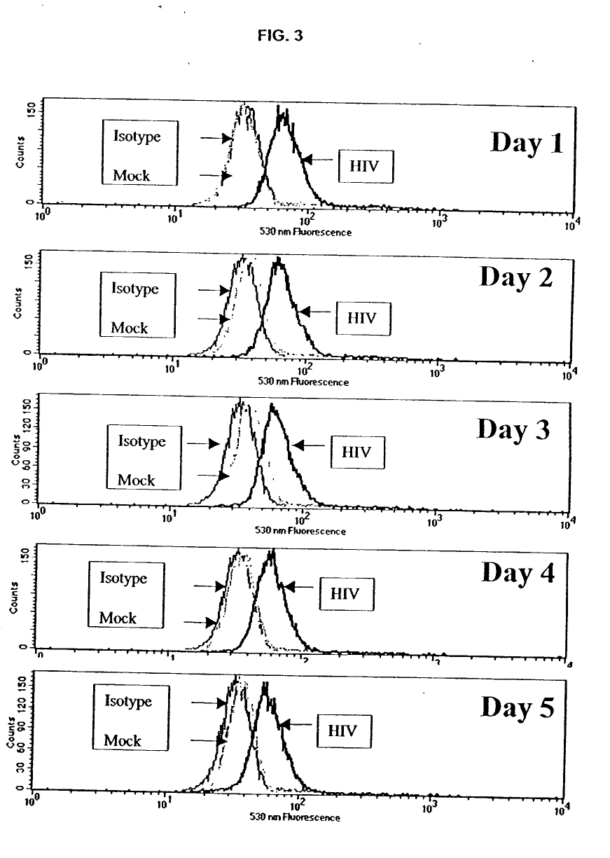

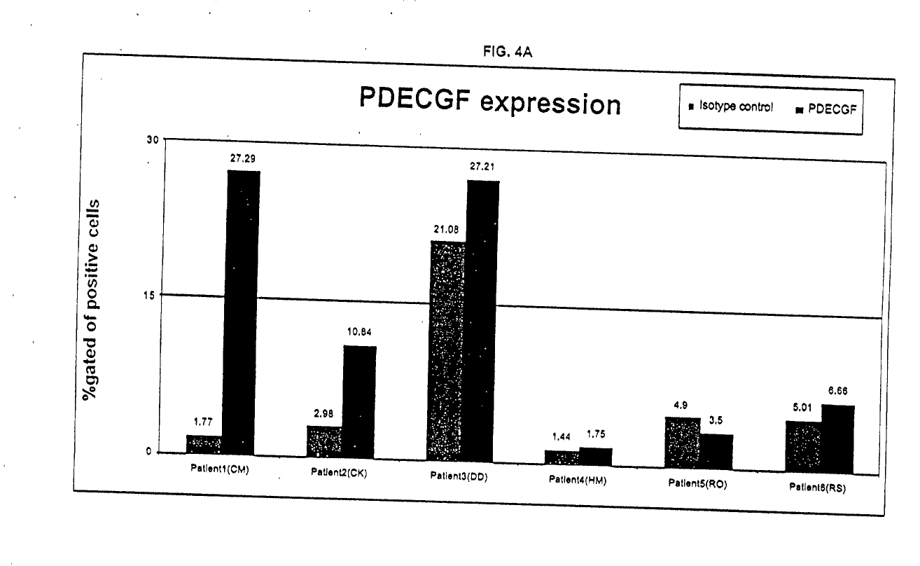

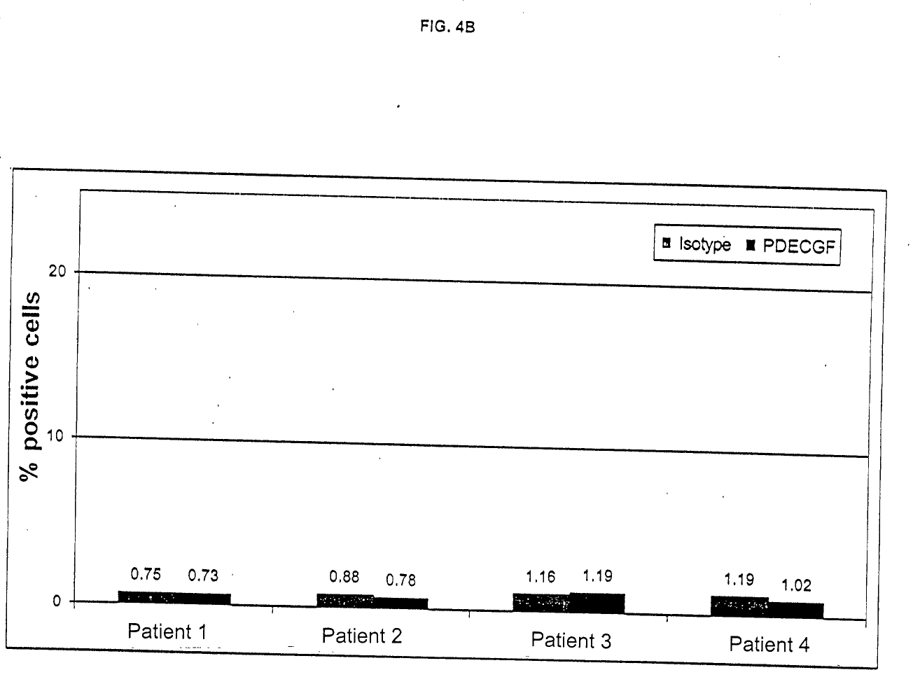

FIG. 1 shows flow cytometry histograms of cells that were either pre-exposed to different concentrations of HIV or only treated with media (mock) and then were stained with either normal goat serum or the goat anti-thymidine phosphorylase. This shows that the higher concentrations of HIV produced higher levels of expression of thymidine phosphorylase, but mock-treated cells were negative for TP. FIG. 2 shows that thymidine phosphorylase can be observed as early as 5 hours after HIV exposure. FIG. 3 shows that the elevated levels of thymidine phosphorylase remain for at least 5 days following HIV contact. HIV+ patients were then evaluated to see if lymphocytes expressing thymidine phosphorylase could be observed. Data from four control patients (FIG. 4A) and six HIV+ patients (FIG. 4B) show that percentages of CD4 cells that were thymidine phosphorylase-positive were elevated in most HIV+ patients, while uninfected persons exhibited no TP-positive CD4 lymphocytes.

### REFERENCES

The following references, to the extent that they provide exemplary procedural or other details supplementary to those set forth herein, are specifically incorporated herein by reference.

Copending application Ser. No. 07/931,811

U.S. Pat. No. 3,817,837

U.S. Pat. No. 3,850,752

U.S. Pat. No. 3,939,350

U.S. Pat. No. 3,996,345

U.S. Pat. No. 4,196,265

U.S. Pat. No. 4,275,149

U.S. Pat. No. 4,277,437

U.S. Pat. No. 4,366,241

U.S. Pat. No. 4,472,509

U.S. Pat. No. 4,659,774

U.S. Pat. No. 4,682,195

U.S. Pat. No. 4,683,202

U.S. Pat. No. 4,816,567

U.S. Pat. No. 4,816,571

U.S. Pat. No. 4,879,236

U.S. Pat. No. 4,938,948

U.S. Pat. No. 4,959,463

U.S. Pat. No. 5,021,236

U.S. Pat. No. 5,141,813

U.S. Pat. No. 5,196,066

U.S. Pat. No. 5,264,566

U.S. Pat. No. 5,428,148

U.S. Pat. No. 5,554,744

U.S. Pat. No. 5,574,146

U.S. Pat. No. 5,587,285

U.S. Pat. No. 5,602,244

U.S. Pat. No. 5,645,897

U.S. Pat. No. 5,674,680

U.S. Pat. No. 5,705,629

U.S. Pat. No. 5,798,213

U.S. Pat. No. 5,843,663

U.S. Pat. No. 5,849,481

U.S. Pat. No. 5,840,873

U.S. Pat. No. 5,843,640

U.S. Pat. No. 5,843,651

U.S. Pat. No. 5,846,708

U.S. Pat. No. 5,846,717

U.S. Pat. No. 5,846,726

U.S. Pat. No. 5,846,729

U.S. Pat. No. 5,849,486

U.S. Pat. No. 5,849,487

U.S. Pat. No. 5,851,772

U.S. Pat. No. 5,853,990

U.S. Pat. No. 5,853,992

U.S. Pat. No. 5,853,993

U.S. Pat. No. 5,856,092

U.S. Pat. No. 5,861,244

U.S. Pat. No. 5,863,732

U.S. Pat. No. 5,863,753

U.S. Pat. No. 5,866,331

U.S. Pat. No. 5,871,986

U.S. Pat. No. 5,900,481

U.S. Pat. No. 5,554,744

U.S. Pat. No. 5,574,146

U.S. Pat. No. 5,587,285

U.S. Pat. No. 5,602,244

U.S. Pat. No. 5,645,897

U.S. Pat. No. 5,674,680

U.S. Pat. No. 5,705,629

U.S. Pat. No. 5,798,213

U.S. Pat. No. 5,843,663

U.S. Pat. No. 5,849,481

U.S. Pat. No. 5,840,873

U.S. Pat. No. 5,843,640

U.S. Pat. No. 5,843,651

U.S. Pat. No. 5,846,708

U.S. Pat. No. 5,846,717

U.S. Pat. No. 5,846,726

U.S. Pat. No. 5,846,729

U.S. Pat. No. 5,849,486

U.S. Pat. No. 5,849,487

U.S. Pat. No. 5,851,772

U.S. Pat. No. 5,853,990

U.S. Pat. No. 5,853,992

U.S. Pat. No. 5,853,993

U.S. Pat. No. 5,856,092

U.S. Pat. No. 5,861,244

U.S. Pat. No. 5,863,732

U.S. Pat. No. 5,863,753

U.S. Pat. No. 5,866,331

U.S. Pat. No. 5,871,986

U.S. Pat. No. 5,900,481

U.S. Pat. No. 5,905,024

U.S. Pat. No. 5,910,407

U.S. Pat. No. 5,912,124

U.S. Pat. No. 5,912,145

U.S. Pat. No. 5,919,626

U.S. Pat. No. 5,919,630

U.S. Pat. No. 5,925,517

U.S. Pat. No. 5,928,862

U.S. Pat. No. 5,928,869

U.S. Pat. No. 5,929,227

U.S. Pat. No. 5,932,413

U.S. Pat. No. 5,935,791

U.S. Pat. No. 5,942,401

U.S. Pat. No. 6,074,646

U.S. Pat. No. 6,197,531

EP 266,032

WO 93/08829

Abbondanzo et al., Breast Cancer Res. Treat. 16:182 (#151), 1990.

Allred et al., Arch Surg, 125(1):107-13, 1990.

Andrieu et al., Infect. Dis. 171:523, 1995.

Antibodies: A Laboratory Manual, Cold Spring Harbor Press, Cold Spring Harbor, N.Y., 1988.

Atherton et al., Biol Reprod, 32(1): 155-171, 1985.

Ausubel et al., In: Current Protocols in Molecular Biology, John, Wiley & Sons, Inc, New York, 1994.

Berberian et al., Science, 261(5128):1588-1591, 1993.

Bragado et al., J. Immunol.159:1619, 1997.

Brown et al., Immunol Ser, 53:69-82, 1990.

Bucy et al., J. Clin. Invest. 103: 1391, 1999.

Chao et al., J. Immunol. 159:1686, 1997.

Chen et al., J. Leukocyte Biol. 72:271-278, 2002.

Chunet al., Nature. 387:183, 1997.

Cleary et al., J. Biol. Chem., 269(29): 18747-9, 1994.

David et al., Biochemistry, 13(5):1014-1021, 1974.

De Jager et al., Semin. Nucl. Med, 23(2):165-179, 1993.

Dholakia et al., J. Biol. Chem., 264(34):20638-20642, 1989.

Dome et al., J. Immunol. 160:2506, 1998.

Doolittle et al., Methods Mol Biol, 109:215-237, 1999.

Embretson et al., Nature. 362:359, 1993.

Finkel et al., Nat. Med. 1:129, 1995.

Ford et al., Semin. Hematol. 6:67, 1969.

Froehler et al., Nucl. Acids. Res. 14:5399-5407, 1986.

Gougeon et al., Semin. Immunol. 5: 187, 1992.

Groux et al., J. Exp. Med 175:331, 1992.

Gulbis and Galand, Hum Pathol, 24(12):1271-85, 1993.

Harper et al., Proc. Natl. Acad. Sci. USA. 83:772, 1986.

Hellerstein et al., Nat. Med 5:83, 1999.

Janeway et al., Immunobiology: The Immune System in Health and Disease. Current Biology Ltd., Garlam Publishing, Inc., New York, 1996.

Janossy et al., Clin. Exp. Immunol. S9:2S7, 1985.

Kang et al., Science, 240(4855): 1034-6, 1988.

Khatoon et al., Ann Neurol, 26(2):210-5, 1989.

King et al., J Biol Chem, 264(17):10210-10218, 1989.

Kirshner et al., J. AIDS. 24:352, 2000.

Kohler et al., Nature, 256(5517):495-7, 1975.

Kohler et al., Methods Enzymol, 178:3-35, 1989.

Kono et al., J. Histochem Cytochem. 49(1): 131-8, 2001.

Kornberg et al., DNA Replication, 2^{nd} Ed., Freeman, San Francisco, 1992.

Kreier et al., Infection, Resistance and Immunity, Harper & Row, New York, 1991.

Lenert et al., Science, 248(4963) 1639-43, 1990.

Mangkornkanok-Mark et al., J. Clin. Exp. Immunol. SS:S81, 1985.

Millstein and Cuello, Nature, 305:537-539, 1983.

Muro-Cacho et al., J. Immunol. 154:5555, 1995.

Nakamura et al., In: Handbook of Experimental Immunology (4^{th} Ed.), Weir, Herzenberg, Blackwell, Herzenberg, (eds). Vol. 1, Chapter 27, Blackwell Scientific Publ., Oxford, 1987.

Neveu et al., Adv. Exp. Med. Biol. 461: 267, 1999.

Nishida et al., Biol. Pharm. Bull. 19(11):1407-11, 1996.

Nygren et al., J. Histochem Cytochem, 30(5):407-412, 1982.

O'Shannessy et al., Anal Biochem, 163(1):204-209, 1987.

Owens et al., J. Biol. Chem. 259: 14843-14848, 1987.

Pain et al., J. Immunol. Methods 40(2):219-30, 1981.

Park et al., Clin. Diagn. Lab. Immunol. 5:583, 1998.

Perelson et al., Science. 271:1 S 82, 1996.

Potter et al., Methods Enzymol, 91:613-633, 1983.

Roederer et al., J. Clin. Invest. 95:2061, 1995.

Sackstein et al., Invest. Med. 43:68, 1995.

Sambrook et. al., In: Molecular Cloning: A Laboratory Manual, 2d Ed., Cold Spring Harbor Laboratory Press, Cold Spring Harbor, N.Y., 1989.

Sambrook et. al., In: Molecular Cloning: A Laboratory Manual, 3rd Ed., Cold Spring Harbor Laboratory Press, Cold Spring Harbor, N.Y., 2001.

Sasso et al., J. Immunol., 142:2778-2783, 1989.

Shorki et al., J. Immunol., 146:936-940, 1991.

Silvermann et al., J. Clin. Invest., 96:417-426, 1995.

Spina et al., J. Virol. 69:2977, 1995.

Suresh et al., Methods in Enzymology, 121:210-228, 1986.

Traunecker et al., EMBO, 10:3655-3659, 1991.

Veazey et al., Science. 280:427, 1998.

Wang et al., J. Immunol. 162:268, 1999.

Wang et al., Virology, 228: 141, 1997.

Wolthers et al., Science. 274:1543, 1996.

Zack et al., J. Viro. 66:1717, 1992.

What is claimed is:

1. A method for evaluating AIDS progression in a patient infected with HIV comprising:
a) obtaining a sample from a patient known to be infected with HIV;
b) assaying the sample for an elevated level of thymidine phosphorylase.

2. The method of claim 1, wherein the sample is a blood sample.

3. The method of claim 2, wherein peripheral blood mononuclear cells are isolated from the blood sample.

4. The method of claim 3, wherein peripheral blood mononuclear cells are assayed for a level of thymidine phosphorylase.

5. The method of claim 1, wherein the level of thymidine phosphorylase is assayed using an antibody directed against a thymidine phosphorylase epitope.

6. The method of claim 5, wherein an ELISA assay is performed on the sample.

7. The method of claim 5, wherein the level of thymidine phosphorylase is assayed immunohistochemically.

8. The method of claim 1, wherein the level of thymidine phosphorylase is assayed by measuring the level of thymidine phosphorylase transcripts.

9. The method of claim 8, wherein the level of thymidine phosphorylase transcripts is measured by amplifying the transcripts.

10. The method of claim 1, wherein the level of thymidine phosphorylase is assayed using mass spectrometry.

11. The method of claim 1, wherein the level of thymidine phosphorylase is assayed by measuring thymidine phosphorylase activity.

12. The method of claim 11, wherein thymidine phosphorylase activity is measured by evaluating the amount of substrate conversion.

13. The method of claim 12, wherein the substrate is thymidine.

14. A method of determining whether a patient is infected with HIV comprising:
a) obtaining a sample from a patient suspected of being infected with HIV;
b) assaying the sample for an elevated level of thymidine phosphorylase.

15. The method of claim 14, wherein the sample is a blood sample.

16. The method of claim 15, wherein peripheral blood mononuclear cells are isolated from the blood sample.

17. The method of claim 16, wherein peripheral blood mononuclear cells are assayed for a level of thymidine phosphorylase.

18. The method of claim 14, wherein the level of thymidine phosphorylase is assayed using an antibody directed against a thymidine phosphorylase epitope.

19. The method of claim 18, wherein an ELISA assay is performed on the sample.

20. The method of claim 18, wherein the level of thymidine phosphorylase is assayed immunohistochemically.

21. The method of claim 14, wherein the level of thymidine phosphorylase is assayed by measuring the level of thymidine phosphorylase transcripts.

22. The method of claim 21, wherein the level of thymidine phosphorylase transcripts is measured by amplifying the transcripts.

23. The method of claim 14, wherein the level of thymidine phosphorylase is assayed using mass spectrometry.

24. The method of claim 14, wherein the level of thymidine phosphorylase is assayed by measuring thymidine phosphorylase activity.

25. The method of claim 24, wherein thymidine phosphorylase activity is measured by evaluating the amount of substrate conversion.

26. The method of claim 25, wherein the substrate is thymidine.

27. A method of evaluating resistance to a thymidine analog AIDS drug in a patient comprising:
a) obtaining a sample from a patient known to be infected with HIV;
b) assaying the sample for a level of thymidine phosphorylase, wherein a elevated level of thymidine phosphorylase is indicative of risk of resistance to the thymidine analog AIDS drug.

28. A method of treating a patient infected with HIV comprising:
a) obtaining a sample from a patient known to be infected with HIV;
b) assaying the sample for a level of thymidine phosphorylase, wherein a elevated level of thymidine phosphorylase is indicative of risk of resistance to the thymidine analog AIDS drug; and
c) administering to the patient an effective amount of an AIDS drug after considering the risk of resistance to the thymidine analog AIDS drug.

29. The method of claim 28, wherein the patient is administered a higher amount of the AIDS drug if the patient is determined to be at risk of resistance to the thymidine analog AIDS drug.

30. A kit for evaluating AIDS progression in a patient comprising, in a suitable container means, an antibody directed against an epitope of human thymidine phosphorylase.

31. The kit of claim 30, further comprising literature indicating a first level of thymidine phosphorylase in a particular sample from a subject not infected with HIV and a second level of thymidine phosphorylase in a particular sample from a subject infected with HIV.

32. An ELISA kit for evaluating AIDS progression in a patient comprising, in a suitable container means, a non-reacting support coupled to an antibody directed against an epitope of human thymidine phosphorylase.
